SEQUENCE LISTING (9 sequences)

SEQ ID NO: 1 - protein, 595 aa, Artificial Sequence
  synthetic sequence
MAGAIASRMSFSSLKRKQPKTFTVRIVTMDAEMEFNCEMKWKGKDLFDLVCRTLGLRETWFFGLQYTIKDTVAWLKMDKKVLDHDVSKEEPVTFHFLAKFYPENAEEELVQEITQHLFFLQVKKQILDEKIYCPPEASVLLASYAVQAKYGDYDPSVHKRGFLAQEELLPKRVINLYQMTPEMWEERITAWYAEHRGRARDEAEMEYLKIAQDLEMYGVNYFAIRNKKGTELLLGVDALGLHIYDPENRLTPKISFPWNEIRNISYSDKEFTIKPLDKKIDVFKFNSSKLRVNKLILQLCIGNHDLFMRRRKADSLEVQQMKAQAREEKARKQMERQRLAREKQMREEAERTRDELERRLLQMKEEATMANEALMRSEETADLLAEKAQITEEEAKLLAQKAAEAEQEMQRIKATAIRTEEEKRLMEQKVLEAEVLALKMAEESERRAKEADQLKQDLQEAREAERRAKQKLLEIATKPTYPPMNPIPAPLPPDIPSFNLIGDSLSFDFKDTDMKRLSMEIEKEKVEYMEKSKHLQEQLNELKTEIEALKLKERETALDILHNENSDRGGSSKHNTIKKLTLQSAKSRVAFFEEL

SEQ ID NO: 2 - DNA, 1799 bp, Artificial Sequence
  synthetic sequence
atggccggcgcgattgcttcccggatgtcattctcctcacttaaaagaaaacagcctaagacttttaccgtgcggatcgtgactatggatgcagagatggaattcaactgcgagatgaagtggaagggaaaggacctcttcgacctcgtctgtcgcaccttgggactgcgggaaacctggttcttcggactgcagtacacaatcaaggacaccgtggcctggctgaaaatggacaagaaggtcctggaccacgacgtgtccaaggaagaacccgtgactttccactttctggccaagttctacccggagaacgctgaggaagaactcgtgcaggagatcactcagcatctgttctttttacaagtcaagaagcaaatcctagatgagaagatctactgcccgccggaagcgtccgtgcttctggcttcctacgcggtgcaggccaaatatggagactacgatccctccgtgcacaagcgcgggttcctggcccaagaggaactgctgcctaagcgcgtgatcaacctgtaccagatgacccccgagatgtgggaggaacgaattacggcttggtacgcggagcatcgcggcagagcacgcgatgaagccgagatggagtacctgaagatcgcgcaggatcttgagatgtacggggtcaactatttcgccatccgcaacaagaagggcaccgaactcctgcttggagtggacgcactcgggctccacatctacgacccggaaaaccgcctgactcccaagatcagcttcccttggaacgaaatcagaaacatttcctactcggataaggaattcacgatcaagccactggacaaaaagattgacgtgttcaagttcaactcgtcgaagctgcgcgtgaacaagctgatactgcaactgtgcattggcaatcacgacctgtttatgcgccggcggaaggccgactcattggaggtccaacagatgaaggcccaggcccgcgaggaaaaggctcgcaagcagatggaacggcagaggctggccagagagaagcagatgcgggaagaggccgaacgcacccgggatgaactggagcgcaggctgctgcagatgaaggaagaagcaaccatggccaacgaagcgctgatgcggagcgaagaaaccgcggacctgttggccgaaaaggctcagatcaccgaggaagaagccaagctccttgcgcaaaaggccgccgaggccgaacaggagatgcagagaatcaaggccaccgccatcagaaccgaagaagagaagcggctcatggaacagaaagtgttggaagccgaggtgctggcgcttaagatggcagaggagtccgagagaagggccaaagaggcagaccagctgaagcaggatctgcaggaagcccgggaagccgagcggcgggcgaagcagaagctcttggaaatcgccaccaaaccgacttacccaccgatgaatcctattcccgcccccctcccccctgacattccctccttcaacctcatcggagactccctgtccttcgacttcaaagacaccgatatgaagcggctgtctatggaaattgaaaaggaaaaggtcgagtacatggagaagtccaagcatctgcaagagcaactcaacgaactcaaaactgagatcgaagctctgaagctgaaggaaagagagactgccctggacattctccataacgaaaactcggacaggggtggtagcagcaagcacaacaccatcaagaagctgactctgcaatcggccaagagccgcgtggcattcttcgaggagctgtaactcgaggatta

SEQ ID NO: 3 - protein, 736 aa, Artificial Sequence
  synthetic sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 308 bp, Artificial Sequence
  synthetic sequence
acatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggtcgcgtctagttattaatgcatactagttaccattgacgtcaataatgacgtatgttcccatagtaacgccaatagggactttccattgacgtcaatgggtggactatttacggtaaactgcccacttggcagtacatcaagtgtatcatatg

SEQ ID NO: 5 - DNA, 741 bp, Artificial Sequence
  synthetic sequence
ctcgagtaaataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcatcgatatc

SEQ ID NO: 6 - DNA, 412 bp, Artificial Sequence
  synthetic sequence
actagttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcactagt

SEQ ID NO: 7 - DNA, 6898 bp, Artificial Sequence
  synthetic sequence
gtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggtcgcgtctagttattaatgcatactagttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcactagttaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggtaccagccaccatggccggcgcgattgcttcccggatgtcattctcctcacttaaaagaaaacagcctaagacttttaccgtgcggatcgtgactatggatgcagagatggaattcaactgcgagatgaagtggaagggaaaggacctcttcgacctcgtctgtcgcaccttgggactgcgggaaacctggttcttcggactgcagtacacaatcaaggacaccgtggcctggctgaaaatggacaagaaggtcctggaccacgacgtgtccaaggaagaacccgtgactttccactttctggccaagttctacccggagaacgctgaggaagaactcgtgcaggagatcactcagcatctgttctttttacaagtcaagaagcaaatcctagatgagaagatctactgcccgccggaagcgtccgtgcttctggcttcctacgcggtgcaggccaaatatggagactacgatccctccgtgcacaagcgcgggttcctggcccaagaggaactgctgcctaagcgcgtgatcaacctgtaccagatgacccccgagatgtgggaggaacgaattacggcttggtacgcggagcatcgcggcagagcacgcgatgaagccgagatggagtacctgaagatcgcgcaggatcttgagatgtacggggtcaactatttcgccatccgcaacaagaagggcaccgaactcctgcttggagtggacgcactcgggctccacatctacgacccggaaaaccgcctgactcccaagatcagcttcccttggaacgaaatcagaaacatttcctactcggataaggaattcacgatcaagccactggacaaaaagattgacgtgttcaagttcaactcgtcgaagctgcgcgtgaacaagctgatactgcaactgtgcattggcaatcacgacctgtttatgcgccggcggaaggccgactcattggaggtccaacagatgaaggcccaggcccgcgaggaaaaggctcgcaagcagatggaacggcagaggctggccagagagaagcagatgcgggaagaggccgaacgcacccgggatgaactggagcgcaggctgctgcagatgaaggaagaagcaaccatggccaacgaagcgctgatgcggagcgaagaaaccgcggacctgttggccgaaaaggctcagatcaccgaggaagaagccaagctccttgcgcaaaaggccgccgaggccgaacaggagatgcagagaatcaaggccaccgccatcagaaccgaagaagagaagcggctcatggaacagaaagtgttggaagccgaggtgctggcgcttaagatggcagaggagtccgagagaagggccaaagaggcagaccagctgaagcaggatctgcaggaagcccgggaagccgagcggcgggcgaagcagaagctcttggaaatcgccaccaaaccgacttacccaccgatgaatcctattcccgcccccctcccccctgacattccctccttcaacctcatcggagactccctgtccttcgacttcaaagacaccgatatgaagcggctgtctatggaaattgaaaaggaaaaggtcgagtacatggagaagtccaagcatctgcaagagcaactcaacgaactcaaaactgagatcgaagctctgaagctgaaggaaagagagactgccctggacattctccataacgaaaactcggacaggggtggtagcagcaagcacaacaccatcaagaagctgactctgcaatcggccaagagccgcgtggcattcttcgaggagctgtaactcgagtaaataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcatcgatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactct

SEQ ID NO: 8 - protein, 590 aa, Artificial Sequence
  synthetic sequence
MAGAIASRMSFSSLKRKQPKTFTVRIVTMDAEMEFNCEMKWKGKDLFDLVCRTLGLRETWFFGLQYTIKDTVAWLKMDKKVLDHDVSKEEPVTFHFLAKFYPENAEEELVQEITQHLFFLQVKKQILDEKIYCPPEASVLLASYAVQAKYGDYDPSVHKRGFLAQEELLPKRVINLYQMTPEMWEERITAWYAEHRGRARDEAEMEYLKIAQDLEMYGVNYFAIRNKKGTELLLGVDALGLHIYDPENRLTPKISFPWNEIRNISYSDKEFTIKPLDKKIDVFKFNSSKLRVNKLILQLCIGNHDLFMRRRKADSLEVQQMKAQAREEKARKQMERQRLAREKQMREEAERTRDELERRLLQMKEEATMANEALMRSEETADLLAEKAQITEEEAKLLAQKAAEAEQEMQRIKATAIRTEEEKRLMEQKVLEAEVLALKMAEESERRAKEADQLKQDLQEAREAERRAKQKLLEIATKPTYPPMNPIPAPLPPDIPSFNLIGDSLSFDFKDTDMKRLSMEIEKEKVEYMEKSKHLQEQLNELKTEIEALKLKERETALDILHNENSDRGGSSKHNTIKKPQAQGRRPICI

SEQ ID NO: 9 - DNA, 1770 bp, Artificial Sequence
  synthetic sequence
atggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagcctcaagcccaaggcagaagacctatctgcatt

(19) United States
(12) Patent Application Publication
(10) Pub. No.: US 2023/0056226 A1
(43) Pub. Date: Feb. 23, 2023

(54) COMPOSITIONS AND METHODS FOR TREATING NEUROFIBROMATIC DISORDERS

(71) Applicant: NF2 Therapeutics, Inc., Cambridge, MA (US)

(72) Inventors: Scott R. Plotkin, Cambridge, MA (US); Michael Wootton, Cambridge, MA (US); David Suhy, San Ramon, CA (US); Shih-chu Kao, Mountain View, CA (US)

(21) Appl. No.: 17/835,566

(22) Filed: Jun. 8, 2022

Related U.S. Application Data
(60) Provisional application No. 63/202,359, filed on Jun. 8, 2021.

(51) Int. Cl. C12N 15/86

(52) U.S. Cl. CPC C12N 15/86; C12N 2750/14143; C12N 2750/14145

(57) ABSTRACT

Compositions and methods for treating neurofibromatic disorders are provided herein, such as expressing Merlin protein or a functional fragment thereof from a viral vector.

### CROSS-REFERENCE TO RELATED APPLICATIONS

This application claims priority to U.S. Provisional Application No. 63/202,359, filed Jun. 8, 2021, which is hereby incorporated by reference in its entirety.

### SEQUENCE LISTING

This application contains a Sequence Listing which has been submitted in ASCII format via EFS-Web and is hereby incorporated by reference in its entirety. The ASCII text file was created on Sep. 21, 2022, it is named 148494.00111_ST25.txt, and it is 32.5 kilobytes in size.

### BACKGROUND

Neurofibromatosis 2 (NF2) is a rare genetic disorder caused by germline mutations in the NF2 gene encoding Merlin, a tumor suppressor protein. These mutations lead to deficiency in Merlin protein which regulates cellular processes including contact inhibition, proliferation, and apoptosis. NF2 is characterized by slow-growing tumors, such as: schwannomas (which arise from Schwann cells), meningiomas (which arise from arachnoid cells), ependymomas (which arise from ependymal cells), as well as juvenile cataracts, and retinal hamartomas.

Typical presentation of NF2 includes bilateral vestibular schwannoma which are multi-lobular masses occurring on the eighth cranial nerve. Vestibular schwannomas cause hearing loss, ringing in the ears, deafness, dizziness, and loss of balance. Patients typically become deafened either because of the effects of tumor growth (including ototoxic effects on the ear leading to death of the cells in the inner ear involved in sensation of sound) or interventions to manage the tumors (including partial or complete removal of the auditory nerve). Left unmanaged, these tumors can eventually compress the brain stem and cause death. Additional symptoms may include disfigurement, facial weakness, headache, and vision loss.

Almost all affected individuals develop these schwannomas by age 30. For individuals with sporadic NF2 (slightly more than half), the average age of diagnosis of NF2 is between 18 to 24 years. The life expectancy of patients with NF2 is 69 years of age, 11 years less than the life expectancy of the general population.

Patients with NF2 have an 80% lifetime probability of developing a meningioma. Meningiomas occur in the meninges, the lining of the brain or spinal cord, and originate in arachnoid cells. The median number of meningiomas these patients develop is three. Over 33% of the tumors are characterized as growing significantly (>1 mm/year). Additionally, meningiomas can also occur in sporadic (non-NF2) patients due to somatic mutations in the NF2 gene. Sporadic meningiomas are the most common brain tumor (— 35% of all brain tumors) and can cause neurological deficits due to compression of brain or spine. Occurrence of meningiomas is the greatest driver of permanent morbidity and mortality in NF2 patients.

There are currently no FDA-approved drugs for meningiomas. The current standard of care includes surgery or radiation therapy. Recurrence is uncommon for meningiomas that are removed completely. In contrast, subtotal resection is associated with high rates of recurrence. Radiation therapy is a reasonable option for patients with non-surgical meningiomas. However, radiation is associated with increased rates of secondary cancers, which is particularly problematic in the context of a tumor suppressor syndrome. For this reason, many clinicians minimize use of radiation in NF2 patients, particularly young patients.

NF2 patients are at increased risk for developing spinal ependymomas. These tumors arise from ependymal cells and typically occur within brainstem or spine. Ependymomas often occur in a “string of pearls” pattern with multiple lesions in the cervical spine. Only a subset of ependymomas will require treatment. The standard approach is surgical resection with radiation reserved for lesions that recur after surgical resection.

In addition to the eighth cranial nerve (CN8), or vestibulocochlear nerve, patients with NF2 commonly develop schwannomas on other cranial nerves including: CN3: oculomotor, CN4: trochlear, CN5: trigeminal, CN6: abducens, CN7: facial, CN9: glossopharyngeal, CN10: vagal, CN11: spinal accessory, CN12: hypoglossal. Damage or loss of function in any of those nerves can cause a wide variety of conditions in the head and neck.

NF2 patients may also develop a number of conditions of the eye including: cataracts, retinal detachment, damage to the nerves of the eye, papilledema (optic disc edema), ocular migraine (retinal migraine), retinitis pigmentosa (RP) (retinal degeneration), combined hamartoma of the retina and RPE, retinal microaneurysms, epiretinal membrane conjunctivitis, physiopedia (severe dry eyes), nystagmus-oscillopsia (ocular flutter/cross), diplopia (double vision), and gaze-evoked tinnitus (GET).

Thus, there is a need for compositions and methods for treating NF2 patients. The present embodiments fulfills these needs as well as others.

### SUMMARY

In some embodiments, an adeno-associated virus (AAV) comprising an adeno associated virus capsid protein and a transgene encoding a full-length Merlin protein or one or more active fragments thereof, such as, but not limited to residues 1-359 of Isoform 1 of Merlin, residues 1-313 of Isoform 1 of Merlin, residues 1-219 of Isoform 1 of Merlin, residues 1-73 of Isoform 1 of Merlin, residues 312-595 of Isoform 1 of Merlin, residues, 479-595 of Isoform 1, residues 503-595 of Isoform 1 of Merlin, or any combination thereof are provided.

In some embodiments, compositions comprising an AAV as provided for herein are provided.

In some embodiments, pharmaceutical compositions comprising an AAV as provided for herein and a pharmaceutically acceptable carrier are provided.

In some embodiments, methods of delivering a Merlin protein to a cell, the method comprising the step of contacting the cell with an AAV as provided for herein.

In some embodiments, methods of treating a subject with NF2, the method comprising administering to the subject with NF2 an AAV as provided for herein.

In some embodiments, methods of inhibiting the growth of a schwannoma, a meningioma, or an ependymoma in a subject, the method comprising administering to an AAV as provided for herein.

In some embodiments, methods of preventing the growth or formation of a schwannoma, ependymoma or a meningioma in a subject, the method comprising administering to the subject an AAV as provided for herein.

In some embodiments, methods of treating a subject with a disorder, or at risk of a disorder, associated with merlin deficiency (e.g., neurofibromatosis type 2, schwannomatosis, or a cancer), the method comprising administering to the subject an AAV as provided for herein.

In some embodiments, a nucleic acid molecule comprising a sequence that is at least 90%, 95%, 96%, 97%, 98%, or 99% identical of SEQ ID NO: 2 is provided herein, or a nucleic acid sequence that encodes the Merlin protein or an active fragment thereof.

In some embodiments, compositions comprising a nucleic acid molecule provided for herein and a carrier are provided.

In some embodiments, methods of producing an AAV as provided herein, the method comprising contacting a cell with a nucleic acid molecule as provided herein to produce the AAV are provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a non-limiting vector (plasmid) map of a recombinant DNA plasmid that can be used to generate AAV particles that comprises a nucleic acid molecule encoding for Merlin protein under a CAG promoter to express MERLIN protein.

FIG. 2 illustrates a non-limiting vector (plasmid) map of a recombinant DNA plasmid that can be used to generate AAV particles that comprises a nucleic acid molecule encoding for Merlin protein under a CAG promoter to express MERLIN protein, this example includes HPRT stuffer sequence, the posttranscriptional regulatory element of HBV as a well as a modified left AAV2 ITR as compared to the plasmid illustrated in FIG. 4. In addition, the bacterial backbone has been modified using kanamycin selection gene to facilitate testing in human clinical studies.

FIG. 3 shows an exemplary Western blot that demonstrates that HEK 293T cells transduced with AAV9-CAG-Merlin-v2 can overexpress Merlin (NF2).

FIG. 4 illustrates exemplary cerebellum tissue sections, which were obtained from cynomolgus macaques at 28 days from administration of AAV9-CAG-Merlin-v2 (2TX-G38) or AAV9-CAG-eGFP (2TX-C10) by intracisternal magna (ICM) injection and are stained against eGFP by immunohistochemistry and demonstrate the cerebellum biodistribution of AAV9-CAG-eGFP.

FIG. 5 shows example enhanced immunofluorescent micrographs of cervical DRG tissue sections stained for the presence of eGFP, obtained from 2 month old Postn-Cre;Nf2^{flox/flox} mice 1 month post administration with AAV9-CAG-GFP by ICM injection, which demonstrate significant biodistribution of eGFP in the DRG of Postn-Cre;Nf2^{flox/flox} mice injected with AAV9-CAG-GFP into the Cisterna Magna.

FIG. 6 shows example enhanced immunofluorescent micrographs of cervical DRG tissue sections stained for the presence of eGFP, obtained from 2 month old Postn-Cre;Nf2^{flox/flox} mice 4 weeks post administration with a low dose of (group 3; 5.6 E12 vg/kg) AAV9-CAG-GFP by ICM injection, and demonstrates GFP expression in the SC surrounding the axonal cells in the DRG of said mice.

FIG. 7 shows example enhanced immunofluorescent micrographs, stained for the presence of eGFP, of tissue sections with nerves distal of the DRG, which were obtained from 2 month old Postn-Cre;Nf2^{flox/flox} mice 4 weeks post administration with low dose (group 3; 5.6 E12 vg/kg) AAV9-CAG-GFP by ICM injection, and demonstrate that eGFP is also observed in the nerves distal of the DRG of NF2 knockout mice dosed by ICM.

FIG. 8 is a graph showing the density of Schwann cell (SC) nuclei (nuclei/μm²) in cross-sections of dorsal root ganglia (DRG) from 6 month old Postn-Cre;Nf2^{flox/flox} mice 5 months from AAV9-CAG-Merlin or AAV9-CAG-GFP administration by ICM injection, which demonstrate that there can be a significant reduction in SC proliferation in NF2 knockout mice administered with AAV9-CAG-Merlin compared to mice injected with the AAV9-CAG-GFP control vector.

FIG. 9 is a non-limiting embodiment of a vector map for pAAV_CAG_Merlin_Kan (SEQ ID NO: 7), which can be used to produce AAV particles comprising a nucleic acid molecule encoding for Merlin under control of a CAG promoter. This vector map, which is similar to FIG. 2, is illustrated with a kanamycin resistant gene to help selection, although other selection genes can be used.

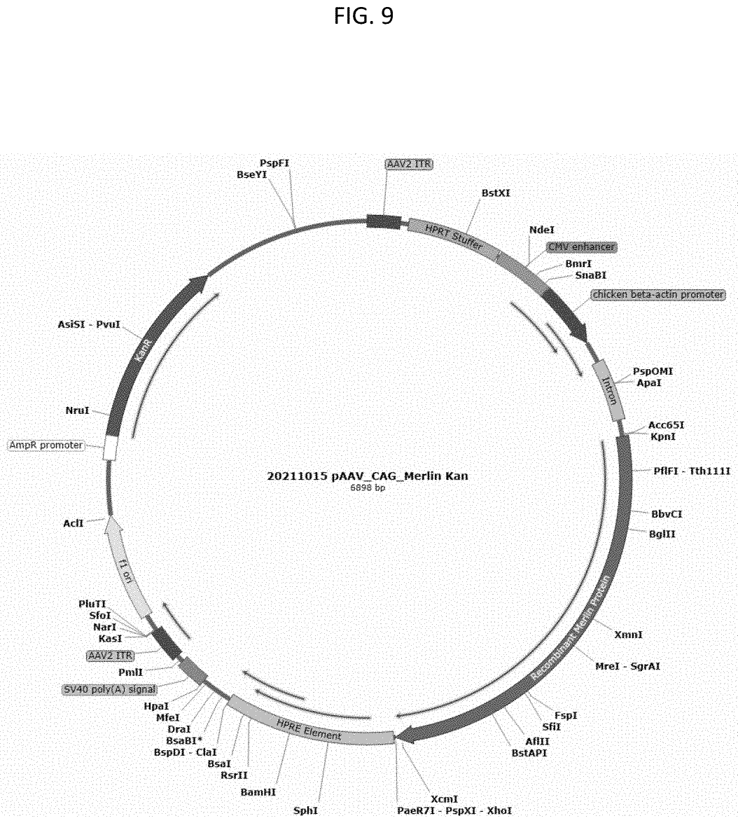

### ENUMERATED EMBODIMENTS

1. An adeno-associated virus (AAV) comprising an AAV capsid protein and a transgene encoding a full-length Merlin protein (e.g. Isoform 1 of Merlin or Isoform 2 of Merlin) or one, or more, active fragments thereof, such as, but not limited to, residues 1-359 of Isoform 1 of Merlin, residues 1-313 of Isoform 1 of Merlin, residues 1-219 of Isoform 1 of Merlin, residues 1-73 of Isoform 1 of Merlin, residues 312-595 of Isoform 1 of Merlin, residues 479-595 of Isoform 1, residues 503-595 of Isoform 1 of Merlin, or combination thereof.

2. The AAV of embodiment 1, wherein the transgene is within AAV inverted terminal repeats.

3. The AAV of embodiments 1 or 2, wherein the transgene is operably linked to regulatory sequences which direct expression of the heterologous gene in a host cell.

4. The AAV of embodiment 3, wherein the regulatory sequences comprise a promoter.

5. The AAV of embodiment 4, wherein the promoter is a constitutive or tissue specific promoter, such as, but not limited to, a neuron or neuronal tissue specific promoter, including, but not limited to, those provided for herein.

6. The AAV of embodiment 5, wherein the promoter is a CAG promoter, a CMV promoter, CBA promoter, or an SV40 promoter.

7. The AAV of any of the preceding embodiments, wherein the capsid protein is an AAV9 capsid protein.

8. The AAV of embodiment 7, wherein the AAV9 capsid protein comprises a protein having the amino acid sequence of:

amino acid residues 1 to 736 of SEQ ID NO: 3;

amino acid residues 138 to 736 of SEQ ID NO: 3; or

amino acid residues 203 to 736 of SEQ ID NO: 3.

9. The AAV of embodiment 7, wherein the AAV9 capsid comprises an amino acid sequence that is at least 80%, 85%, 90%, 95%, 96%, 97%, 98%, or 99% identical to amino acid residues 203 to 736 of SEQ ID NO: 3.

10. The AAV of any of the preceding embodiments, wherein the Merlin protein encoded by the transgene comprises a sequence that is at least 90%, 95%, 96%, 97%, 98%, or 99% identical or homologous to a sequence of SEQ ID NO: 1 or SEQ ID NO: 8.

11. The AAV of any of the preceding embodiments, wherein the Merlin protein encoded by the transgene comprises a sequence of SEQ ID NO: 1 or SEQ ID NO: 8, or an active fragment thereof.

12. The AAV of any of the preceding embodiments, wherein the transgene comprises a nucleic acid molecule encoding a protein comprising the sequence of SEQ ID NO: 1 of SEQ ID NO: 8, or an active fragment thereof.

13. The AAV of embodiment 12, wherein the nucleic acid molecule encoding the Merlin protein comprises a nucleic acid sequence of SEQ ID NO: 2, SEQ ID NO: 9, or a codon optimized version of each of the foregoing.

14. The AAV of embodiment 2, wherein the AAV inverted terminal repeats are AAV-2 inverted terminal repeats.

15. The AAV of embodiment 14, wherein an AAV2 inverted terminal repeat comprises a sequence of SEQ ID NO: 4.

16. The AAV of any of the preceding embodiments, wherein the AAV comprises a nucleic acid molecule stuffer sequence upstream of the transgene and downstream of the 5′ (left) AAV ITR.

17. The AAV of embodiment 16, wherein the stuffer sequence comprises a sequence of SEQ ID NO: 6.

18. The AAV of any of the preceding embodiments, wherein the AAV comprises a nucleotide intron sequence that is downstream of the transgene and upstream of the right (3′) AAV (e.g., AAV2) ITR.

19. The AAV of embodiment 18, wherein the intron sequence is a HPRE sequence.

20. The AAV of embodiment 19, wherein the HPRE sequence comprises a sequence of SEQ ID NO: 5.

21. A composition comprising the AAV of any one of embodiments 1-20 and a physiologically compatible carrier.

22. A pharmaceutical composition comprising the AAV of any one of embodiments 1-20 and a pharmaceutically acceptable carrier.

23. A method of delivering a Merlin protein to a cell, the method comprising the step of contacting the cell with the AAV of any one of embodiments 1-20 or the composition of embodiments 21 or 22.

24. A method of treating a subject with NF2, the method comprising administering to the subject with NF2 the AAV of any one of embodiments 1-20 or the composition of embodiments 21 or 22.

25. The method of embodiment 24, wherein the administration is intraventricular, intracisternal, intrathecal, intrastriatal, intrapleural, intramuscular, intravitreal, intravenous, or intratumoral.

26. A method of inhibiting the growth of a schwannoma, a meningioma, or an ependymoma in a subject, the method comprising administering to the subject the AAV of any one of embodiments 1-20 or the composition of embodiments 21 or 22.

27. The method of embodiment 26, wherein the subject is a subject with NF2 or NF2 deficiency disorder, such as schwannomatosis.

28. The method of embodiment 26, wherein the administration is intrathecal, intravenous, intraventricular, intrastriatal, intrapleural, intramuscular, intracisternal, or intratumoral.

29. A method of preventing the growth or formation of a schwannoma, ependymoma or a meningioma in a subject, the method comprising administering to the subject the AAV of any one of embodiments 1-20 or the composition of embodiments 21 or 22.

30. The method of embodiment 29, wherein the subject is a subject with NF2 or NF2 deficiency disorder, such as schwannomatosis.

31. The method of embodiment 30, wherein the administration is intrathecal, intravenous, intracisternal, or intratumoral.

32. A method of treating a subject with a disorder, or at risk of a disorder, associated with merlin deficiency (e.g., neurofibromatosis type 2, schwannomatosis, or a cancer), the method comprising administering to the subject the AAV of any one of embodiments 1-20 or the composition of embodiments 21 or 22.

33. The method of embodiment 32, wherein the disorder is neurofibromatosis type 2, schwannomatosis, schwannomas (e.g., vestibular schwannomas); a cancer (e.g., a hematological cancer (e.g., juvenile myelomonocytic leukemia), a leukemia (e.g., adult acute lymphoblastic leukemia, childhood acute lymphoblastic leukemia, adult acute myeloid leukemia, childhood acute myeloid leukemia, chronic lymphocytic leukemia, chronic myelogenous leukemia, or hairy cell leukemia); a lymphoma (e.g., AIDS-related lymphoma, cutaneous T-cell lymphoma, adult hodgkin lymphoma, childhood hodgkin lymphoma, adult non-hodgkin lymphoma, childhood non-hodgkin lymphoma, primary central nervous system lymphom, sézary syndrome, cutaneous T-cell lymphoma, cutaneous waldenstrom macroglobulinemia); a chronic myeloproliferative disorder; langerhans cell histiocytosis; multiple myeloma/plasma cell neoplasm; a myelodysplastic syndrome; a myelodysplastic/myeloproliferative neoplasm); an ovarian cancer (e.g., ovarian serous carcinoma); breast cancer, invasive breast carcinoma; or a neurocutaneous disorder; (e.g., mesothelioma, peritoneal mesothelioma, skin squamous cell carcinoma, cancer of the urinary tract, thyroid cancer, stomach cancer, schwannoma, renal cell carcinoma, cancer of the pituitary, ovarian cancer, meningioma, melanoma, lung cancer (e.g., squamous cell carcinoma, non-small cell lung cancer, mixed lung cancer, lung adenocarcinoma), liver cancer, large intestine cancer, hepatocellular carcinoma, acute myelogenous leukemia (AML), aerodigestive tract cancer (squamous cell carcinoma), bladder cancer, bone cancer (e.g., bone sarcoma), colorectal carcinoma, ependymoma, colorectal carcinoma, endometrium (mixed adenosquamous carcinoma), or a glioma, or cataracts, retinal detachment, damage to the nerves of the eye, papilledema (optic disc edema), ocular migraine (retinal migraine), retinitis pigmentosa (RP) (retinal degeneration), combined hamartoma of the retina and RPE, retinal microaneurysms, epiretinal membrane conjunctivitis, physiopedia (severe dry eyes), nystagmus-oscillopsia (ocular flutter/cross), diplopia (double vision), or gaze-evoked tinnitus (GET).

34. The method of any of embodiments 23-33, wherein the AAV is administered at least every 6 months, 9 months, 12 months, 15 months, 18 months, 21 months, 2 years, three years, four years, five years, six years, or more.

35. The method of any of embodiments 23-34, wherein the AAV is administered intravenously, intradermally, subcutaneously, intrathecally, systemically, intracisternally, intraventricularly, intraparnchymally, intrapleurally, intrastriatally, intramuscularly, intravitreally, or locally.

36. A nucleic acid molecule comprising a sequence that is at least 90%, 95%, 96%, 97%, 98%, or 99% identical, or is identical, to SEQ ID NO: 2.

37. The nucleic acid molecule of embodiment 30, wherein the nucleic acid molecule is an isolated nucleic acid molecule.

38. The nucleic acid molecule of embodiments 36 or 37, wherein the nucleic acid molecule comprises a sequence of SEQ ID NO: 2.

39. The nucleic acid molecule of any one of embodiments 36-39, wherein the nucleic acid molecule comprises AAV inverted terminal repeats.

40. The nucleic acid molecule of embodiment 39, wherein the sequence of SEQ ID NO: 2 is bound within the AAV inverted terminal repeats.

41. The nucleic acid molecule of embodiments 39 or 40, wherein the AAV inverted terminal repeats are AAV2 inverted terminal repeats.

42. The nucleic acid molecule of embodiment 41, wherein one of the AAV2 inverted terminal repeat (e.g., the 3′ AAV ITR) comprises a sequence of SEQ ID NO: 4.

43. The nucleic acid molecule of any one of embodiments 36-42, wherein the nucleic acid comprising SEQ ID NO: 2 is operably linked to regulatory sequences which direct expression of the protein encoded by SEQ ID NO: 2.

44. The nucleic acid molecule of embodiment 44, wherein the regulatory sequences comprises a promoter.

45. The nucleic acid molecule of embodiment 44, wherein the promoter is a constitutive promoter or a tissue specific promoter.

46. The nucleic acid molecule of embodiments 37 or 38, wherein the promoter is a CAG promoter, a CMV promoter, or an SV40 promoter.

47. The nucleic acid molecule of any one of embodiments 36-46, wherein the nucleic acid molecule comprises a stuffer sequence upstream of SEQ ID NO: 2 transgene and downstream of the 5′ (left) AAV ITR.

48. The nucleic acid molecule of embodiment 47, wherein the stuffer sequence comprises a sequence of SEQ ID NO: 6.

49. The nucleic acid molecule of any one of embodiments 36-48, wherein the nucleic acid molecule comprises a nucleotide intron sequence that is downstream of SEQ ID NO: 2 and upstream of the right (3′) AAV (e.g. AAV2) ITR.

50. The nucleic acid molecule of embodiment 49, wherein the intron sequence is a HPRE intron sequence.

51. The nucleic acid molecule of embodiment 50, wherein the HPRE intron sequence comprises a sequence of SEQ ID NO: 5.

52. The nucleic acid molecule of any one of embodiments 36-51, wherein the molecule is a plasmid.

53. A composition comprising the nucleic acid molecule of any one of embodiments 36-52 and a carrier.

54. The composition of embodiment 53, wherein the carrier is a transfection reagent.

55. A method of producing an AAV particle of any one of embodiments 1-20, the method comprising contacting (e.g. transfecting or electroporating) a cell with a nucleic acid molecule of any one of embodiments 36-52 or the composition of embodiments 53 or 54 to produce the AAV.

56. The method of embodiment 55, wherein the cell is of an AAV packaging cell line.

57. The method of embodiment 56, wherein the AAV packaging cell line is an AAV9 packaging cell line.

58. A cultured host cell comprising a recombinant nucleic acid molecule encoding an AAV capsid protein and a recombinant nucleic acid molecule encoding Merlin protein.

59. The cell of embodiment 58, wherein the capsid protein is the AAV9 capsid protein.

60. The cell of embodiments 58 or 59, wherein the nucleic acid molecule encoding the capsid protein encoding a protein that is at least 80%, 85%, 90%, 95%, 96%, 97%, 98%, or 99% identical, or is identical, to a protein having the amino acid sequence of: amino acid residues 1 to 736 of SEQ ID NO: 3; amino acid residues 138 to 736 of SEQ ID NO: 3; or amino acid residues 203 to 736 of SEQ ID NO: 3.

61. The cell of anyone of embodiments 58-60, wherein the recombinant nucleic acid molecule encoding the Merlin protein encodes a protein that is at least 90%, 95%, 96%, 97%, 98%, or 99% identical or homologous, or is identical, to a sequence of SEQ ID NO: 1, SEQ ID NO: 8, or an active fragment thereof.

62. The cell of anyone of embodiments 58-61, wherein the recombinant nucleic acid molecule encoding the Merlin protein encodes a protein that comprises a sequence of SEQ ID NO: 1, SEQ ID NO: 8, or an active fragment thereof.

63. The cell of anyone of embodiments 58-62, wherein the recombinant nucleic acid molecule encoding the Merlin protein comprises a sequence that is at least 90%, 95%, 96%, 97%, 98%, or 99% identical to SEQ ID NO: 2 or SEQ ID NO: 9.

64. The cell of anyone of embodiments 58-63, wherein the recombinant nucleic acid molecule encoding the Merlin protein comprises a sequence of SEQ ID NO: 2 of SEQ ID NO: 9.

65. The cell of anyone of embodiments 58-64, wherein the recombinant nucleic acid molecule is a plasmid.

### DETAILED DESCRIPTION

Embodiments provided for herein are directed to, in part, compositions, methods and other embodiments for treating NF2 disease or conditions, tumors, or disorders related to Merlin protein deficiency.

Because of the reduction of functional Merlin, which plays a critical role as a tumor suppressor protein, leads to Neurofibromatosis 2 or a condition, tumor, or a disorder related to Merlin deficiency, restoring Merlin activity by delivering functional copies of the NF2 gene may lead to an effective treatment.

Accordingly, in some embodiments, a recombinant virus is provided that comprises a transgene that encodes for the Merlin protein. The type of virus used can be any virus that when used to infect a cell or a subject will lead to the expression of the transgene and the Merlin protein, or any portion thereof, in the cell or in the subject. In some embodiments, the portion is an N-terminal fragment of the Merlin protein (Tikoo et. al., An Anti-Ras Function of Neurofibromatosis Type 2 Gene Product (NF2/Merlin), The Journal of Biological Chemistry, Vol. 269, No 38, Issue of Sep. 23, 1994 pp. 23387-23390; Cui et. al., The NF2 tumor suppressor merlin interacts with Ras and RasGAP, which may modulate Ras signaling, Oncogene, Vol. 38, 2019, pp. 6370-6381). In some embodiments, the N-terminal fragment of Merlin encoded comprises or consists of amino acid residues 1-359 of Isoform 1 of Merlin. In some embodiments, the N-terminal fragment of Merlin encoded comprises or consists of amino acid residues 1-313 of Isoform 1 of Merlin. In some embodiments, the N-terminal fragment of Merlin encoded comprises or consists of amino acid residues 1-219 of Isoform 1 of Merlin. In some embodiments, the N-terminal fragment of Merlin encoded comprises or consists of amino acid residues 1-73 of Isoform 1 of Merlin. In some embodiments, the portion is a C-terminal fragment of the Merlin protein (Cui et. al., The NF2 tumor suppressor merlin interacts with Ras and RasGAP, which may modulate Ras signaling, Oncogene, Vol. 38, 2019, pp. 6370-6381). In some embodiments, the C-terminal fragment of Merlin encoded comprises or consists of amino acid residues 312-595 of Isoform 1 of Merlin. In some embodiments, the C-terminal fragment of Merlin encoded comprises or consists of amino acid residues 479-595 of Isoform 1 of Merlin. In some embodiments, the C-terminal fragment of Merlin encoded comprises or consists of amino acid residues 503-595 of Isoform 1 of Merlin.

In some embodiments, the “Active Fragment”, as used herein, is a fragment of wild type Merlin that has biological activity. In some embodiments, the “Isoform”, as used herein, is an alternative splicing variant of wild type Merlin.

In some embodiments, the virus is an adeno-associated virus (AAV). AAV has many naturally occurring serotypes that can be used. In some embodiments, the AAV serotype is AAV2, AAV3, AAV4, AAV5, AAV6, AAV7, AAV8, AAV9, or AAV-DJ. The serotype refers to the capsid protein that is encoded by the particular strain of the AAV. In addition, the proteins which comprise that capsid proteins of different AAV serotypes can be engineered, or modified from the naturally occurring serotypes, to have enhanced properties. Enhanced properties as a result of capsid engineering may include, but are not limited to, ease of scalable manufacture of AAV, reduced immunogenicity, altered tissue tropism and/or provide advantageous properties for therapeutic application. Therefore, in some embodiments, an AAV is provided that comprises an AAV capsid protein and a transgene encoding a Merlin protein. Methods of making AAV are known in the art and can be found in, for example, U.S. Pat. No. 7,906,111, which is hereby incorporated by reference in its entirety. The transgene can be incorporated in what can be referred to as a minigene that comprises AAV inverted terminal repeats as well as sequences that encode for the Merlin protein. The sequences of the transgene can also be operably linked to regulatory elements or sequences that direct the expression of the Merlin protein in a host cell.

The regulatory elements can include conventional control elements which are operably linked to the transgene in a manner which permits its transcription, translation and/or expression in a cell transfected with the plasmid vector or infected with the virus. As used herein, “operably linked” sequences include both expression control sequences that are contiguous with the gene of interest and expression control sequences that act in trans or at a distance to control the gene of interest.

Expression control sequences can include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, CAG promoter (Miyazaki et al, Gene. 79 (2): 269-77; Niwa et al., Gene. 108 (2): 193-9), the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), the simian virus 40 (SV40) promoter, the dihydrofolate reductase promoter, the beta-actin promoter, the phosphoglycerol kinase (PGK) promoter, the human elongation factor 1 alpha (EF1) promoter (Qin, J. Y., Zhang, L., Clift, K. L., Hulur, I., Xiang, A. P., Ren, B. Z., et al. (2010). Systematic comparison of constitutive promoters and the doxycycline-inducible promoter. PLoS One 5 (5), e10611. doi: 10.1371/journal.pone.0010611), and the human ubiquitin C promoter (UBC), (Qin, J. Y., Zhang, L., Clift, K. L., Hulur, I., Xiang, A. P., Ren, B. Z., et al. (2010). Systematic comparison of constitutive promoters and the doxycycline-inducible promoter. PLoS One 5 (5), e10611. doi: 10.1371/journal.pone.0010611). Inducible promoters allow regulation of gene expression and can be regulated by exogenously supplied compounds. environmental factors such as temperature, or the presence of a specific physiological state, e.g., acute phase, a particular differentiation state of the cell, or in replicating cells only. Inducible promoters and inducible systems are available from a variety of commercial sources. Many other systems have been described and can be readily selected by one of skill in the art. Examples of inducible promoters regulated by exogenously supplied compounds, include, the zinc-inducible sheep metallothionein (MT) promoter, the dexamethasone (Dex)-inducible mouse mammary tumor virus (MMTV) promoter, the T7 polymerase promoter system (International Patent Publication No. WO 98/10088); the ecdysone insect promoter (No et al, Proc. Natl. Acad. Sci. USA, 93:3346-3351 (1996)), the tetracycline-repressible system (Gossen et al, Proc. Natl. Acad Sci. USA, 89:5547-5551 (1992)), the tetracycline-inducible system (Gossen et al, Science, 268:1766-1769 (1995), see also Harvey et al, Curr. Opin. Chem. BioL, 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Other types of inducible promoters which may be useful in this context are those which are regulated by a specific physiological state, e.g., temperature, acute phase, a particular differentiation state of the cell, or in replicating cells only.

In some embodiments, the promoter is the CAG promoter. In some embodiments, the promoter is the SV40 promoter. In some embodiments, the promoter is the CMV promoter. In some embodiments, the promoter is a constitutive promoter. In some embodiments, the promoter is not a tissue or cell specific promoter. In some embodiments, the promoter is a tissue or cell specific promoter. In some embodiments, the promoter is not an inducible promoter.

In some embodiments, the native promoter for the Merlin product will be used. The native promoter can be used when it is desired that expression of Merlin should mimic the native expression. The native promoter may be used when expression of the transgene must be regulated temporally or developmentally, or in a tissue-specific manner, or in response to specific transcriptional stimuli. In some embodiments, other native expression control elements, such as enhancer elements, polyadenylation sites or Kozak consensus sequences may be included in the promoter sequence in order to more closely mimic the native expression.

In some embodiments, the transgene includes a gene operably linked to a tissue-specific promoter. In some embodiments, the promoter is a neuron or neuronal tissue specific promoter. Examples of such promoters, include, but are not limited to, a neuron-specific enolase (NSE) promoter (Andersen et al., Cell. Mol. Neurobiol., 13:503-15 (1993)), a neurofilament light-chain gene (Piccioli et al., Proc. Natl. Acad Sci. USA, 88:5611-5 (1991)), the neuron-specific VGF gene (Piccioli et al., Neuron, 15:373-84 (1995)), or a human synapsin promoter (Syn1), (Kugler, S., Kilic, E., and Bahr, M. (2003). Human synapsin 1 gene promoter confers highly neuron-specific long-term transgene expression from an adenoviral vector in the adult rat brain depending on the transduced area. Gene Ther. 10 (4), 337-347. doi: 10.1038/sj.gt.3301905, Kugler, S., Meyn, L., Holzmuller, H., Gerhardt, E., Isenmann, S., Schulz, J. B., et al. (2001). Neuron-specific expression of therapeutic proteins: evaluation of different cellular promoters in recombinant adenoviral vectors. Mol. Cell Neurosci. 17 (1), 78-96. doi: 10.1006/mcne.2000.0929, McLean, J. R., Smith, G. A., Rocha, E. M., Hayes, M. A., Beagan, J. A., Hallett, P. J., et al. (2014). Widespread neuron-specific transgene expression in brain and spinal cord following synapsin promoter-driven AAV9 neonatal intracerebroventricular injection. Neurosci. Lett. 576, 73-78. doi: 10.1016/j. neulet.2014.05.044), or chicken beta actin (CBA), or CBh (a hybrid of CBA) (Gray et. al., Optimizing Promoters for Recombinant Adeno-Associated Virus-Mediated Gene Expression in the Peripheral and Central Nervous System Using Self-Complementary Vectors, HUMAN GENE THERAPY 22:1143-1153 (September 2011) a Mary Ann Liebert, Inc.), or MeCp2 (Gray et. al., Optimizing Promoters for Recombinant Adeno-Associated Virus-Mediated Gene Expression in the Peripheral and Central Nervous System Using Self-Complementary Vectors, HUMAN GENE THERAPY 22:1143-1153 (September 2011) a Mary Ann Liebert, Inc.), or PDGFβ (Ingusci et. al., Gene Therapy Tools for Brain Diseases, Selene Ingusci, Frontiers in Pharmacology, Jul. 1, 2019) astrocyte specific such as GFAP (Smith-Arica, J. R., Morelli, A. E., Larregina, A. T., Smith, J., Lowenstein, P. R., and Castro, M. G. (2000). Cell-type-specific and regulatable transgenesis in the adult brain: adenovirus-encoded combined transcriptional targeting and inducible transgene expression. Mol. Ther. 2 (6), 579-587. doi: 10.1006/mthe.2000.0215, Lee, Y., Messing, A., Su, M., and Brenner, M. (2008). GFAP promoter elements required for region-specific and astrocyte-specific expression. Glia 56 (5), 481-493. doi: 10.1002/glia.20622), oligodendrocyte specific such as human myelin associated glycoprotein (MAG), (von Jonquieres, G., Mersmann, N., Klugmann, C. B., Harasta, A. E., Lutz, B., Teahan, O., et al. (2013). Glial promoter selectivity following AAV-delivery to the immature brain. PLoS One 8 (6), e65646. doi: 10.1371/journal.pone.0065646), or myelin basic promoter (MBP) (von Jonquieres, G., Frohlich, D., Klugmann, C. B., Wen, X., Harasta, A. E., Ramkumar, R., et al. (2016). Recombinant human myelin-associated glycoprotein promoter drives selective AAV-mediated transgene expression in oligodendrocytes. Front. Mol. Neurosci. 9, 13. doi: 10.3389/fnmol.2016.00013), microglia specific such as F4/80 (Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), or CD68 (Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), glutamatergic neuron specific such as phosphate-activated glutaminase promoter (PAG), (Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j. brainres.2007.01.125), or vascular glutamate transporter promoter (vGLUT), (Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j. brainres.2007.01.125), and GABAergic neuron specific such as glutamic acid decarboxylase promoter (GAD)(Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j. brainres.2007.01.125), among others. Other promoters can also be used, such as, but not limited to where expression in skeletal muscle is desired, a promoter active in muscle should be used. These include the promoters from genes encoding skeletal P-actin, myosin light chain 2A, dystrophin, muscle creatine kinase, as well as synthetic muscle promoters with activities higher than naturally-occurring promoters (see Li et al., Nat. Biotech., 17:241-245 (1999)). Other tissue specific promoters include, promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein el al., Mol. Biol. Rep., 24:185-96 (1997)); bone sialoprotein (Chen et aL, J. Bone Miner. Res., 11:654-64 (1996)), lymphocytes (CD2, Hansal et al., J. Immunol., 161:1063-8 (1998); immunoglobulin heavy chain; T cell receptor chain),

The combination of the transgene product, the promoter/enhancer regulatory sequences, and 5′ and 3′ AAV ITRs can collectively be referred to as a “minigene” for ease of reference herein.

In some embodiments, the capsid used is taken from AAV9. In some embodiments, the AAV9 capsid comprises a group of viral proteins (VP1, VP2, VP3, etc.) having the amino acid sequence of: amino acid residues 1 to 736 of SEQ ID NO: 3; amino acid residues 138 to 736 of SEQ ID NO: 3; or amino acid residues 203 to 736 of SEQ ID NO: 3. In some embodiments, the AAV9 capsid comprises a group of viral proteins (VP1, VP2, VP3, etc.) having the amino acid sequence of amino acid residues 1 to 736 of SEQ ID NO: 3. In some embodiments, the AAV9 capsid comprises a group of viral proteins (VP1, VP2, VP3, etc.) having the amino acid sequence of amino acid residues 138 to 736 of SEQ ID NO: 3. In some embodiments, the AAV9 capsid comprises a group of viral proteins (VP1, VP2, VP3, etc.) having the amino acid sequence of amino acid residues 203 to 736 of SEQ ID NO: 3. In some embodiments, the AAV9 capsid comprises an amino acid sequence that is at least 80%, 85%, 90%, 95%, 96%, 97%, 98%, or 99% identical to amino acid residues 1 to 736 of SEQ ID NO: 3; amino acid residues 138 to 736 of SEQ ID NO: 3; or amino acids 203 to 736 of SEQ ID NO: 3. In some embodiments, the AAV9 capsid comprises an amino acid sequence that is at least 80%, 85%, 90%, 95%, 96%, 97%, 98%, or 99% identical to amino acid residues 1 to 736 of SEQ ID NO: 3. In some embodiments, the AAV9 capsid comprises an amino acid sequence that is at least 80%, 85%, 90%, 95%, 96%, 97%, 98%, or 99% identical to amino acid residues 138 to 736 of SEQ ID NO: 3. In some embodiments, the AAV9 capsid comprises an amino acid sequence that is at least 80%, 85%, 90%, 95%, 96%, 97%, 98%, or 99% identical to amino acid residues 203 to 736 of SEQ ID NO: 3.

In some embodiments, the recombinant AAV genome encodes for the Merlin protein. In some embodiments, the Merlin is isoform 1. In some embodiments, the Merlin is isoform 2.

In some embodiments, the Merlin protein encoded by the transgene comprises a sequence that is at least 90%, 95%, 96%, 97%, 98%, or 99% identical or homologous to a sequence of SEQ ID NO: 1 (isoform 1). In some embodiments, the Merlin protein encoded by the transgene comprises a sequence of SEQ ID NO: 1. In some embodiments, the recombinant AAV genome encodes for one or more active fragments of the Merlin protein. In some embodiments, the one or more active fragments include, but not limited to, residues 1-359 of SEQ ID NO: 1, residues 1-313 of SEQ ID NO: 1, residues 1-219 of SEQ ID NO: 1, residues 1-73 of SEQ ID NO: 1, residues 312-595 of SEQ ID NO: 1, residues 479-595 of SEQ ID NO: 1, residues 503-595 of SEQ ID NO: 1, or any combination thereof. In some embodiments, the one or more active fragments include, but not limited to, residues 1-359 of SEQ ID NO: 1. In some embodiments, the one or more active fragments include, but not limited to, residues 1-313 of SEQ ID NO: 1. In some embodiments, the one or more active fragments include, but not limited to, residues 1-219 of SEQ ID NO: 1. In some embodiments, the one or more active fragments include, but not limited to, residues 1-73 of SEQ ID NO: 1. In some embodiments, the one or more active fragments include, but not limited to, residues 312-595 of SEQ ID NO: 1. In some embodiments, the one or more active fragments include, but not limited to, residues 479-595 of SEQ ID NO: 1. In some embodiments, the one or more active fragments include, but not limited to, residues 503-595 of SEQ ID NO: 1.

The active fragments, in some embodiments, can comprises a sequence that is at least 90%, 95%, 96%, 97%, 98%, or 99% identical or homologous to such fragments.

In some embodiments, the transgene comprises a nucleic acid molecule encoding a protein comprising a sequence of SEQ ID NO: 1, or an active fragment thereof. In some embodiments, the nucleic acid molecule comprises a nucleic acid sequence of SEQ ID NO: 2, or a sequence encoding the active fragments thereof. In some embodiments, the nucleic acid molecule comprises a nucleic acid sequence that is least 90%, 95%, 96%, 97%, 98%, or 99% identical to SEQ ID NO: 2, or to the sequence encoding the active fragments thereof.

Also provided herein, are variants of polypeptides or nucleic acid molecules described herein, which include polypeptides or nucleic acid molecules having at least 75%, 80%, 85%, 90%, 95%, 96%, 97%, 98% or 99% identity with the amino acid sequences or nucleic acid molecules provided for herein or of the wild-type sequences. For example, variants of Merlin (NF2) include polypeptides having at least 75%, 80%, 85%, 90%, 95%, 96%, 97%, 98% or 99% identity or sequence homology with the amino acid sequence of SEQ ID NO: 1, or an active fragment thereof. For example, variants of nucleic acid molecules encoding Merlin (NF2) include nucleic acid molecules having at least 75%, 80%, 85%, 90%, 95%, 96%, 97%, 98% or 99% identity with the SEQ ID NO: 2, or encoding an active fragment thereof are provided.

In some embodiments, the Merlin protein encoded by the transgene comprises a sequence that is at least 90%, 95%, 96%, 97%, 98%, or 99% identical or homologous to a sequence of SEQ ID NO: 8 (isoform 2). In some embodiments, the Merlin protein encoded by the transgene comprises a sequence of SEQ ID NO: 8. In some embodiments, the recombinant AAV genome encodes for one or more active fragments of the Merlin protein. The active fragments, in some embodiments, can comprises a sequence that is at least 90%, 95%, 96%, 97%, 98%, or 99% identical or homologous to such fragments.

In some embodiments, the transgene comprises a nucleic acid molecule encoding a protein comprising a sequence of SEQ ID NO: 8, or an active fragment thereof. In some embodiments, the nucleic acid molecule comprises a nucleic acid sequence of SEQ ID NO: 9, or a sequence encoding the active fragments thereof. In some embodiments, the nucleic acid molecule comprises a nucleic acid sequence that is least 90%, 95%, 96%, 97%, 98%, or 99% identical to SEQ ID NO: 9, or to the sequence encoding the active fragments thereof.

Also provided herein, are variants of polypeptides or nucleic acid molecules described herein, which include polypeptides or nucleic acid molecules having at least 75%, 80%, 85%, 90%, 95%, 96%, 97%, 98% or 99% identity with the amino acid sequences or nucleic acid molecules provided for herein or of the wild-type sequences. For example, variants of Merlin (NF2) include polypeptides having at least 75%, 80%, 85%, 90%, 95%, 96%, 97%, 98% or 99% identity or sequence homology with the amino acid sequence of SEQ ID NO: 8, or an active fragment thereof. For example, variants of nucleic acid molecules encoding Merlin (NF2) include nucleic acid molecules having at least 75%, 80%, 85%, 90%, 95%, 96%, 97%, 98% or 99% identity with the SEQ ID NO: 9, or encoding an active fragment thereof are provided.

Calculations of “identity” or “sequence homology” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

Variants of polypeptides described herein can be those having amino acid modifications (e.g., deletions, additions or substitutions, such as conservative substitutions) from the wild-type amino acid sequence of the polypeptide, or any active fragment thereof as described above. For example, a variant of Merlin can differ by at least 1, 2, 3, 4, 5 but not more than 50, 40, 30, 20, 15 or 10 amino acids from Merlin (SEQ ID NO: 1 or SEQ ID NO: 8). The sequence may also have conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Also provided herein are isolated nucleic acid molecules. An “isolated” protein or nucleic acid molecule refers to a purified protein or a nucleic acid molecule that is removed from at least 90% of at least one component of a natural sample from which the isolated protein or nucleic acid molecule can be obtained. Proteins can be “of at least” a certain degree of purity if the species or population of species of interest is at least 5, 10, 25, 50, 75, 80, 90, 92, 95, 98, or 99% pure on a weight-weight basis.

As provided for herein, the recombinant AAV can comprise AAV inverted terminal repeats (ITR). In some embodiments, the inverted terminal repeats used are the AAV-2 inverted terminal repeats. ITRs from other AAV serotypes may also be used and this is merely a non-limiting example.

In addition to AAV being the vehicle for delivering Merlin protein (e.g. by delivering DNA encoding for the Merlin protein), other vehicles can be used, such as adenovirus, retroviruses, lentiviruses, and the like. Therefore, in some embodiments, a recombinant virus for gene delivery is provided that encodes for Merlin protein, or an active fragment thereof, and expresses Merlin, or an active protein fragment thereof, in the infected cell. The AAV can also be pseudotyped with other viral structural or capsid proteins (non-AAV) to help specify the cell type that is infected by the AAV. Therefore, in some embodiments, the AAV is a pseudotyped AAV.

Compositions comprising the AAV or virus encoding for Merlin, or an active fragment thereof, are also provided. In some embodiments, the composition comprises a recombinant virus encoding for Merlin, or an active fragment thereof, and a physiologically compatible carrier. In some embodiments, the composition is a pharmaceutical composition and comprises a pharmaceutically acceptable carrier.

By “pharmaceutically acceptable”, it is meant the carrier, diluent or excipient must be compatible with the other ingredients of the recombinant virus and not significantly deleterious to the recipient thereof.

In some embodiments, the composition or pharmaceutical composition comprises an effective amount of the virus. This can also be referred to as a therapeutically effective amount. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the therapeutic to elicit a desired response in the individual. A therapeutically effective amount is also one in which any toxic or detrimental effects as a result of the composition are outweighed by therapeutically beneficial effects.

As provided herein, the compositions can comprise pharmaceutically acceptable vehicles, carrier, or excipients. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, and additional pharmaceutical agents.

In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Also provided herein are nucleic acid molecules comprising a sequence that is at least 90%, 95%, 96%, 97%, 98%, or 99% identical of SEQ ID NO: 2. In some embodiments, the nucleic acid molecule is an isolated nucleic acid molecule. In some embodiments, the nucleic acid molecule comprises a sequence of SEQ ID NO: 2. In some embodiments, SEQ ID NO: 2 can be used to distinguish between endogenous mRNA encoding for Merlin protein and the mRNA that is produced from the AAV encoding for the Merlin protein. SEQ ID NO: 2 is a non-limiting example of a sequence that is distinct from the native mRNA and another sequence that can be distinguished from the native sequence can be used. Accordingly, in some embodiments, methods of detecting a Merlin mRNA encoded by the AAV are provided. In some embodiments, the method comprises contacting a sample with a probe that is specific to the AAV encoding mRNA and does not bind with the native mRNA and detecting the AAV mRNA when the probe binds to interacts with the AAV encoding mRNA. In some embodiments, the method comprises performing RT-PCR on a sample with primers that are specific to the AAV encoding mRNA to detect the AAV encoded mRNA. In some embodiments, the primers are specific to the AAV encoded mRNA and do not bind or interact with a native mRNA. As used herein, the term “native mRNA” refers to a RNA that encoded by the cell's or organisms original DNA that has not been modified, i.e. wild-type that has not been genetically manipulated. This type of assay, can be used, for example, in a subject that has been treated with the AAV compositions provided for herein to determine expression of the AAV encoding merlin protein, which is distinct from the native protein.

In some embodiments, the nucleic acid molecule comprises AAV inverted terminal repeats. In some embodiments, the sequence of SEQ ID NO: 2 is bound within the AAV inverted terminal repeats. In some embodiments, the AAV inverted terminal repeats are the AAV2 inverted terminal repeats. In some embodiments, the nucleic acid comprising SEQ ID NO: 2 is operably linked to regulatory sequences which direct expression of the protein encoded by SEQ ID NO: 2. In some embodiments, the regulatory sequences comprise a promoter. In some embodiments, the promoter is as described herein. In some embodiments, the promoter is a constitutive promoter or a tissue specific promoter. In some embodiments, the promoter is a CAG promoter, a CMV promoter, or an SV40 promoter. In some embodiments, the promoter is a CAG promoter. In some embodiments, the promoter is a CMV promoter. In some embodiments, the promoter is an SV40 promoter. In some embodiments, the nucleic acid molecule is a plasmid.

In some embodiments, non-viral vectors are used. The compositions are provided that comprise the nucleic acid molecules described herein. In some embodiments, the compositions comprise a carrier. In some embodiments, the carrier is a transfection reagent or a reagent to facilitate the delivery of the nucleic acid molecule to a cell. In some embodiments, the transfection reagent is a lipid based transfection reagent. In some embodiments, the carrier is an electroporation agent.

The nucleic acid molecules provided for herein can be used to produce an AAV that encodes for the Merlin protein. Methods of producing AAV are known to one of skill in the art and any method can be used. A non-limiting example is provided in U.S. Pat. No. 7,906,111, which is hereby incorporated by reference. For example, the minigene can be carried on any suitable vector, e.g., a plasmid, which is delivered to a host cell. The plasmids may be engineered such that they are suitable for replication and, optionally, integration in prokaryotic cells, mammalian cells, or both. In some embodiments, these plasmids (or other vectors carrying the 5′ AAV ITR-heterologous molecule-3′ AAV ITR) contain sequences permitting replication of the minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. In some embodiments, the molecule carrying the minigene is transfected into the cell, where it may exist transiently. Alternatively, the minigene (carrying the 5′ AAV ITR-heterologous molecule-3′ ITR) may be stably integrated into the genome of the host cell, either chromosomally or as an episome. In some embodiments, the minigene may be present in multiple copies, optionally in head-to-head, head-to-tail, or tail-to-tail concatamers. Suitable transfection techniques are known and may readily be utilized to deliver the minigene to the host cell.

In some embodiments, the AAV comprises a nucleic acid molecule stuffer sequence upstream of the transgene and downstream of the 5′ (left) AAV ITR. In some embodiments, the stuffer sequence comprises a sequence of SEQ ID NO: 6. In some embodiments, the AAV comprises a nucleotide intron sequence that is downstream of the transgene and upstream of the right (3′) AAV (e.g., AAV2) ITR. In some embodiments, the intron sequence is a HPRE sequence, such as those described herein. In some embodiments, the HPRE sequence comprises a sequence of SEQ ID NO: 5.

In some embodiments, when delivering the vector comprising the minigene by transfection, the vector is delivered in an amount from about 5 μg to about 100 μg DNA, about 10 μg to about 50 μg DNA to about 1×10⁴ cells to about 1×10¹³ cells, or about 1×10⁵ cells. However, the relative amounts of vector DNA to host cells may be adjusted, taking into consideration such factors as the selected vector, the delivery method and the host cells selected.

The host cell itself for producing the AAV may be selected from any biological organism, including prokaryotic (e.g., bacterial) cells, and eukaryotic cells, including, insect cells, yeast cells and mammalian cells. In some embodiments, the host cells are selected from among any mammalian species, including, without limitation, cells such as A549, WEH1, 3T3, 10T1/2, BHK, MDCK, COS 1, COS 7, BSC 1, BSC 40, BMT 10, VERO. W138, HeLa, 293 cells (which express functional adenoviral E1), Saos-2, C2C12, L cells, HT1080, HepG2 and primary fibroblast, hepatocyte and myoblast cells derived from mammals including human, monkey, mouse, rat, rabbit, and hamster. The requirements for the cell used is that it not carry any adenovirus gene other than E1, E2a and/or E4 ORF6, which is done to avoid homologous recombination of a contaminating virus during the production of AAV; and it is capable of infection or transfection of DNA and expression of the transfected DNA.

In some embodiments, the host cell is stably transfected with the capsid protein, such as AAV9 capsid. It can also have the rep protein being produced from the same cell. Alternatively, these proteins can be encoded for on separate plasmids that encode for the viral proteins and are transfected into the cell at the same time or sequentially into the host cell to produce the AAV encoding for Merlin. For example, one host cell that could be used is a host cell stably transformed with the sequences encoding rep and cap, and which is transfected with the adenovirus E1, E2a, and E4ORF6 DNA and a construct carrying the minigene as described above. Stable rep and/or cap expressing cell lines, such as B-50 (International Patent Application Publication No. WO 99/15685), or those described in U.S. Pat. No. 5,658,785, may also be similarly employed. Another example of a host cell is one that contains the minimum adenoviral DNA which is sufficient to express E4 ORF6. Yet other cell lines can be constructed using the AAV9 cap sequences provided for herein.

The preparation of a host cell involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In some embodiments, standard transfection techniques are used, e.g., CaPO₄ transfection, lipid based transfection, or electroporation, and/or infection by hybrid adenovirus/AAV vectors into cell lines such as the human embryonic kidney cell line HEK 293 (a human kidney cell line containing functional adenovirus E1 gene which provides trans-acting E1 proteins).

The AAV9 capsid protein can also be used to pseudotype other viruses encoding for Merlin. Thus, they could be used in other rAAV and non-rAAV vector systems. Such vectors systems may include. e.g., lentiviruses, retroviruses, poxviruses, vaccinia viruses, and adenoviral system, among others.

Accordingly, in some embodiments, methods of producing an AAV as provided for herein are provided. In some embodiments, the method comprises contacting a cell with a nucleic acid molecule as provided herein or a composition of the same to produce the AAV. In some embodiments, the cell is an AAV packaging cell line. In some embodiments, the AAV packaging cell line is an AAV9 packaging cell line, such as those described herein or in U.S. Pat. No. 7,906,111, which is hereby incorporated by reference in its entirety.

In some embodiments, a cultured host cell is provided that comprises a recombinant nucleic acid molecule encoding an AAV capsid protein and a recombinant nucleic acid molecule encoding Merlin protein. In some embodiments, the capsid protein is the AAV9 capsid protein or variants thereof as described herein.

In some embodiments, methods of delivering a Merlin protein to a cell are provided. In some embodiments, the methods comprise contacting the cell with an AAV or virus encoding for Merlin, such as those described herein. In some embodiments, the methods comprise non-viral delivery methods, such as needle injection, ballistic DNA injection, sono-poration, photo-poration, magneto-fection, hydro-poration, or electro-poration, transfection, and any combination thereof. In some embodiments, the non-viral methods comprise a liposome or a polymer carrier, and a combination thereof, for delivery of the genetic material encoding Merlin protein.

In some embodiments, methods of treating a subject with NF2, or other merlin deficiency are provided, the methods comprising administering to the subject with NF2, or other merlin deficiency, a virus or an AAV encoding for Merlin, such as those provided herein. The method of administration can be any suitable method, such as those described herein. In some embodiments, the administration is intrathecal, intravenous, intrapleural, or intratumoral.

In some embodiments, methods of inhibiting the growth of a schwannoma, a meningioma, mesothelioma, or an ependymoma in a subject are provided. In some embodiments, the method comprises administering to the subject virus or an AAV encoding for Merlin, such as those provided herein. The method of administration can be any suitable method, such as those described herein. In some embodiments, the administration is intrathecal, intracisternal intravenous, intrapleural, or intratumoral.

In some embodiments, methods of treating a subject with NF2 are provided. In some embodiments, the subject is a subject with NF2. In some embodiments, the subject is a subject with a NF2 deficiency. As used herein, the term “NF2 deficiency” refers to a condition or disorder in a subject that is caused by a mutated or inactivated Merlin protein. This can also be referred to as a “Merlin deficiency.” These conditions can be the various types of tumors or disorders provided herein. In some embodiments, the disorder is as provided herein. In some embodiments, the disorder is cataracts, retinal detachment, damage to the nerves of the eye, papilledema (optic disc edema), ocular migraine (retinal migraine), retinitis pigmentosa (RP) (retinal degeneration), combined hamartoma of the retina and RPE, retinal microaneurysms, epiretinal membrane conjunctivitis, physiopedia (severe dry eyes), nystagmus-oscillopsia (ocular flutter/cross), diplopia (double vision), or gaze-evoked tinnitus (GET). In some embodiments, a method of treating a subject with a disorder, or at risk of a disorder, associated with merlin deficiency (e.g., neurofibromatosis type 2, schwannomatosis, or a cancer), is provided, and the method comprises administering to the subject an AAV, such as those provided herein.

In some embodiments, methods of preventing the growth or formation of a spinal schwannomas or a meningioma in a subject are provided. In some embodiments, the method comprises administering to the subject virus or an AAV encoding for Merlin, such as those provided herein. The method of administration can be any suitable method, such as those described herein. In some embodiments, the administration is intrathecal, intravenous, or intratumoral. In some embodiments, the subject is a subject with NF2.

In some embodiments, methods of treating a subject with a disorder, or at risk of a disorder, associated with merlin (e.g., neurofibromatosis type 2, schwannomatosis, or a cancer) are provided. In some embodiments, the method comprises administering to the subject virus or an AAV encoding for Merlin, such as those provided herein. The method of administration can be any suitable method, such as those described herein. In some embodiments, the administration is intrathecal, intravenous, intrapleural, or intratumoral. In some embodiments, the subject is a subject with NF2. In some embodiments, the disorder is neurofibromatosis type 2, schwannomatosis, schwannomas (e.g., vestibular schwannomas); a cancer (e.g., a hematological cancer (e.g., juvenile myelomonocytic leukemia), a leukemia (e.g., adult acute lymphoblastic leukemia, childhood acute lymphoblastic leukemia, adult acute myeloid leukemia, childhood acute myeloid leukemia, chronic lymphocytic leukemia, chronic myelogenous leukemia, or hairy cell leukemia); a lymphoma (e.g., AIDS-related lymphoma, cutaneous T-cell lymphoma, adult hodgkin lymphoma, childhood hodgkin lymphoma, adult non-hodgkin lymphoma, childhood non-hodgkin lymphoma, primary central nervous system lymphom, sézary syndrome, cutaneous T-cell lymphoma, cutaneous waldenstrom macroglobulinemia); a chronic myeloproliferative disorder; langerhans cell histiocytosis; multiple myeloma/plasma cell neoplasm; a myelodysplastic syndrome; a myelodysplastic/myeloproliferative neoplasm); an ovarian cancer (e.g., ovarian serous carcinoma); breast cancer; breast invasive ductal carcinoma, or a neurocutaneous disorder; (e.g., mesothelioma, pleural mesothelioma, pleural epithelioid mesothelioma, cutaneous melanoma, cancer of the urinary tract, thyroid cancer, thyroid anaplastic carcinoma, stomach cancer, schwannoma, renal cell carcinoma, papillary renal cell carcinoma, cancer of the pituitary, ovarian cancer, meningioma, melanoma, lung cancer (e.g., squamous cell carcinoma, mixed lung cancer, lung adenocarcinoma), liver cancer, large intestine cancer, hepatocellular carcinoma, acute myelogenous leukemia (AML), aerodigestive tract cancer (squamous cell carcinoma), bladder cancer, bladder urothelial carcinoma, bone cancer (e.g., bone sarcoma), colorectal carcinoma, ependymoma, colorectal carcinoma, colorectal adenocarcinoma, pancreatic adenocarcinoma, endometrium (mixed adenosquamous carcinoma), endometrial endometrioid adenocarcinoma, or a glioma, or a conventional glioblastoma multiforme. In some embodiments, the disorder is cataracts, retinal detachment, damage to the nerves of the eye, papilledema (optic disc edema), ocular migraine (retinal migraine), retinitis pigmentosa (RP) (retinal degeneration), combined hamartoma of the retina and RPE, retinal microaneurysms, epiretinal membrane conjunctivitis, physiopedia (severe dry eyes), nystagmus-oscillopsia (ocular flutter/cross), diplopia (double vision), or gaze-evoked tinnitus (GET).

As used herein and in the appended claims, the singular forms “a”, “an” and “the” include plural reference unless the context clearly dictates otherwise.

As used herein, the terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

The terms “co-administration” or the like, are meant to encompass administration of the selected therapeutic agents to a single patient, and are intended to include treatment regimens in which the agents are administered by the same or different route of administration or at the same or different time.

The term “subject” or “patient” as used herein includes, but is not limited to, humans and non-human vertebrates such as wild, domestic, and farm animals. In certain embodiments, the subject or patient described herein is an animal. In certain embodiments, the subject or patient is a mammal. In certain embodiments, the subject is a human. In certain embodiments, the subject or patient is a non-human animal. In certain embodiments, the subject or patient is a non-human mammal. In certain embodiments, the subject or patient is a domesticated animal, such as a dog, cat, cow, pig, horse, sheep, or goat. In certain embodiments, the subject or patient is a companion animal such as a dog or cat. In certain embodiments, the subject or patient is a livestock animal such as a cow, pig, horse, sheep, or goat. In certain embodiments, the subject or patient is a zoo animal. In another embodiment, the subject or patient is a research animal such as a rodent, dog, or non-human primate. In certain embodiments, the subject or patient is a non-human transgenic animal such as a transgenic mouse or transgenic pig.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; remission (whether partial or total), whether detectable or undetectable, or enhancement or improvement of the condition, disorder or disease; and prevention of disorder or disease manifestation. Treatment includes eliciting a clinically significant response without excessive levels of side effects. Treatment also includes prolonging survival as compared to expected survival if not receiving treatment.

“Encoding” refers to the inherent property of specific sequences of nucleotides in a polynucleotide, such as a gene, a cDNA, or an mRNA or viral RNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, the expression vector is the alphaviruses as described herein.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

### Formulations and Routes for Administration to Patients

Where clinical applications or methods are contemplated, it will be necessary to prepare pharmaceutical compositions—expression constructs, viruses, expression vectors, fused proteins, transfected or transduced cells, in a form appropriate for the intended application. Generally, this will entail preparing compositions that are essentially free of pyrogens, except for the viruses or plasmids provided for herein, as well as other impurities that could be harmful to humans or animals. [00.161] The recombinant nucleic acids, virus products or pharmaceutical compositions thereof, may be delivered, for example at doses of about 1-5 million particles per dose. Vials or other containers may be provided containing the product, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

One may generally desire to employ appropriate salts and buffers when recombinant nucleic acid, viruses, or viral products are introduced into a patient. The phrase “pharmaceutically or pharmacologically acceptable” refers to molecular entities and compositions that do not produce a significant adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

Upon formulation, solutions can be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

The compositions may be formulated for aerosolized delivery to a subject. For aerosol delivery, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents.

Delivery systems of the disclosure that deliver the polynucleotides of the disclosure to a desired cell of a subject are not limited to the viruses of the disclosure. In some embodiments, the delivery vector comprises a virus as provided for herein. These can be used in methods of treating disorders or conditions, such as those described herein.

In some embodiments, the therapeutic is administered at least every 6 months, 9 months, 12 months, 15 months, 18 months, 21 months, 2 years, three years, four years, five years, six years or more. In some embodiments, the therapeutic is administered intravenously, intradermally, subcutaneously, intrathecally, which includes which includes intraventricularly, intracisternally, or injections into lumbar locations, intra-cisterna magnally, intrapleurally, intravitreally, subretinally, intramuscularly, systemically, or locally, such as intratumoral injection.

### Kits

Additionally, certain components or embodiments of these recombinant nucleic acids, viruses, or pharmaceutical compositions thereof, can be provided in a kit. For example, any of the recombinant nucleic acids, or, viruses, can be provided frozen or refrigerated and packaged as a kit, alone or along with separate containers of any of the other agents from the pre-conditioning or post-conditioning steps, and optional instructions for use.

Some embodiments are also directed to any of the aforementioned compositions in a kit. In some embodiments, the kit may comprise ampoules, disposable syringes, capsules, vials, tubes, or the like. In some embodiments, the kit may comprise a single dose container or multiple dose containers comprising the topical formulation of embodiments herein. In some embodiments, each dose container may contain one or more unit doses. In some embodiments, the kit comprises a pre-filled syringe containing a pharmaceutical composition. In some embodiments, the kit may include an applicator. In some embodiments, the kits include all components needed for the stages of conditioning/treatment. In some embodiments, the cellular compositions may have preservatives or be preservative-free (for example, in a single-use container). In some embodiments, the recombinant nucleic acids, virus products may be prepared and frozen or refrigerated at a desired stage, suitable for shipping to a hospital or treatment center.

Various sequences are referenced herein, such sequence can comprise, as applicable, one or more of the following sequences:

MERLIN ISOFORM 1 (SEQ ID NO: 1):
MAGAIASRMSFSSLKRKQPKTFTVRIVTMDAEMEFNCEMKWKGKDLFDLVCRTLGLRETWFFGLQ
YTIKDTVAWLKMDKKVLDHDVSKEEPVTFHFLAKFYPENAEEELVQEITQHLFFLQVKKQILDEK
IYCPPEASVLLASYAVQAKYGDYDPSVHKRGFLAQEELLPKRVINLYQMTPEMWEERITAWYAEH
RGRARDEAEMEYLKIAQDLEMYGVNYFAIRNKKGTELLLGVDALGLHIYDPENRLTPKISFPWNE
IRNISYSDKEFTIKPLDKKIDVFKFNSSKLRVNKLILQLCIGNHDLFMRRRKADSLEVQQMKAQA
REEKARKQMERQRLAREKQMREEAERTRDELERRLLQMKEEATMANEALMRSEETADLLAEKAQI
TEEEAKLLAQKAAEAEQEMQRIKATAIRTEEEKRLMEQKVLEAEVLALKMAEESERRAKEADQLK
QDLQEAREAERRAKQKLLEIATKPTYPPMNPIPAPLPPDIPSFNLIGDSLSFDFKDTDMKRLSME
IEKEKVEYMEKSKHLQEQLNELKTEIEALKLKERETALDILHNENSDRGGSSKHNTIKKLTLQSA
KSRVAFFEEL
NUCLEIC ACID SEQUENCE ENCODING MELRIN ISOFORM 1 (SEQ ID NO: 2)
ATGGCCGGCGCGATTGCTTCCCGGATGTCATTCTCCTCACTTAAAAGAAAACAGCCTAAGACTTT
TACCGTGCGGATCGTGACTATGGATGCAGAGATGGAATTCAACTGCGAGATGAAGTGGAAGGGAA
AGGACCTCTTCGACCTCGTCTGTCGCACCTTGGGACTGCGGGAAACCTGGTTCTTCGGACTGCAG
TACACAATCAAGGACACCGTGGCCTGGCTGAAAATGGACAAGAAGGTCCTGGACCACGACGTGTC
CAAGGAAGAACCCGTGACTTTCCACTTTCTGGCCAAGTTCTACCCGGAGAACGCTGAGGAAGAAC
TCGTGCAGGAGATCACTCAGCATCTGTTCTTTTTACAAGTCAAGAAGCAAATCCTAGATGAGAAG
ATCTACTGCCCGCCGGAAGCGTCCGTGCTTCTGGCTTCCTACGCGGTGCAGGCCAAATATGGAGA
CTACGATCCCTCCGTGCACAAGCGCGGGTTCCTGGCCCAAGAGGAACTGCTGCCTAAGCGCGTGA
TCAACCTGTACCAGATGACCCCCGAGATGTGGGAGGAACGAATTACGGCTTGGTACGCGGAGCAT
CGCGGCAGAGCACGCGATGAAGCCGAGATGGAGTACCTGAAGATCGCGCAGGATCTTGAGATGTA
CGGGGTCAACTATTTCGCCATCCGCAACAAGAAGGGCACCGAACTCCTGCTTGGAGTGGACGCAC
TCGGGCTCCACATCTACGACCCGGAAAACCGCCTGACTCCCAAGATCAGCTTCCCTTGGAACGAA
ATCAGAAACATTTCCTACTCGGATAAGGAATTCACGATCAAGCCACTGGACAAAAAGATTGACGT
GTTCAAGTTCAACTCGTCGAAGCTGCGCGTGAACAAGCTGATACTGCAACTGTGCATTGGCAATC
ACGACCTGTTTATGCGCCGGCGGAAGGCCGACTCATTGGAGGTCCAACAGATGAAGGCCCAGGCC
CGCGAGGAAAAGGCTCGCAAGCAGATGGAACGGCAGAGGCTGGCCAGAGAGAAGCAGATGCGGGA
AGAGGCCGAACGCACCCGGGATGAACTGGAGCGCAGGCTGCTGCAGATGAAGGAAGAAGCAACCA
TGGCCAACGAAGCGCTGATGCGGAGCGAAGAAACCGCGGACCTGTTGGCCGAAAAGGCTCAGATC
ACCGAGGAAGAAGCCAAGCTCCTTGCGCAAAAGGCCGCCGAGGCCGAACAGGAGATGCAGAGAAT
CAAGGCCACCGCCATCAGAACCGAAGAAGAGAAGCGGCTCATGGAACAGAAAGTGTTGGAAGCCG
AGGTGCTGGCGCTTAAGATGGCAGAGGAGTCCGAGAGAAGGGCCAAAGAGGCAGACCAGCTGAAG
CAGGATCTGCAGGAAGCCCGGGAAGCCGAGCGGCGGGCGAAGCAGAAGCTCTTGGAAATCGCCAC
CAAACCGACTTACCCACCGATGAATCCTATTCCCGCCCCCCTCCCCCCTGACATTCCCTCCTTCA
ACCTCATCGGAGACTCCCTGTCCTTCGACTTCAAAGACACCGATATGAAGCGGCTGTCTATGGAA
ATTGAAAAGGAAAAGGTCGAGTACATGGAGAAGTCCAAGCATCTGCAAGAGCAACTCAACGAACT
CAAAACTGAGATCGAAGCTCTGAAGCTGAAGGAAAGAGAGACTGCCCTGGACATTCTCCATAACG
AAAACTCGGACAGGGGTGGTAGCAGCAAGCACAACACCATCAAGAAGCTGACTCTGCAATCGGCC
AAGAGCCGCGTGGCATTCTTCGAGGAGCTGTAACTCGAGGATTA
AAV9 CAPSID (SEQ ID NO: 3)
MAADGYLPDWLEDNLSEGIREWWALKPGAPQPKANQQHQDNARGLVLPGYKYLGPGNGLDKGEPV
NAADAAALEHDKAYDQQLKAGDNPYLKYNHADAEFQERLKEDTSFGGNLGRAVFQAKKRLLEPLG
LVEEAAKTAPGKKRPVEQSPQEPDSSAGIGKSGAQPAKKRLNFGQTGDTESVPDPQPIGEPPAAP
SGVGSLTMASGGGAPVADNNEGADGVGSSSGNWHCDSQWLGDRVITTSTRTWALPTYNNHLYKQI
SNSTSGGSSNDNAYFGYSTPWGYFDFNRFHCHFSPRDWQRLINNNWGFRPKRLNFKLFNIQVKEV
TDNNGVKTIANNLTSTVQVFTDSDYQLPYVLGSAHEGCLPPFPADVFMIPQYGYLTLNDGSQAVG
RSSFYCLEYFPSQMLRTGNNFQFSYEFENVPFHSSYAHSQSLDRLMNPLIDQYLYYLSKTINGSG
QNQQTLKFSVAGPSNMAVQGRNYIPGPSYRQQRVSTTVTQNNNSEFAWPGASSWALNGRNSLMNP
GPAMASHKEGEDRFFPLSGSLIFGKQGTGRDNVDADKVMITNEEEIKTTNPVATESYGQVATNHQ
SAQAQAQTGWVQNQGILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQYTSNYYKSNNVEFAV
NTEGVYSEPRPIGTRYLTRNL
Left-ITR sequence that can be used as an alternative ITR replaces
the original fragment within pAV-CAG-Merlin. (SEQ ID NO: 4)
ACATGTCCTGCAGGCAGCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGCCAACTC
CATCACTAGGGGTTCCTGCGGCCGGTCGCGTCTAGTTATTAATGCATACTAGTTACCATTGACGT
CAATAATGACGTATGTTCCCATAGTAACGCCAATAGGGACTTTCCATTGACGTCAATGGGTGGAC
TATTTACGGTAAACTGCCCACTTGGCAGTACATCAAGTGTATCATATG
HPRE fragment (SEQ ID NO: 5)
CTCGAGTAAATAACAGGCCTATTGATTGGAAAGTTTGTCAACGAATTGTGGGTCTTTTGGGGTTT
GCTGCCCCTTTTACGCAATGTGGATATCCTGCTTTAATGCCTTTATATGCATGTATACAAGCAAA
ACAGGCTTTTACTTTCTCGCCAACTTACAAGGCCTTTCTCAGTAAACAGTATATGACCCTTTACC
CCGTTGCTCGGCAACGGCCTGGTCTGTGCCAAGTGTTTGCTGACGCAACCCCCACTGGTTGGGGC
TTGGCCATAGGCCATCAGCGCATGCGTGGAACCTTTGTGTCTCCTCTGCCGATCCATACTGCGGA
ACTCCTAGCCGCTTGTTTTGCTCGCAGCAGGTCTGGAGCAAACCTCATCGGGACCGACAATTCTG
TCGTACTCTCCCGCAAGTATACATCGTTTCCATGGCTGCTAGGCTGTGCTGCCAACTGGATCCTG
CGCGGGACGTCCTTTGTTTACGTCCCGTCGGCGCTGAATCCCGCGGACGACCCCTCCCGGGGCCG
CTTGGGGCTCTACCGCCCGCTTCTCCGTCTGCCGTACCGTCCGACCACGGGGCGCACCTCTCTTT
ACGCGGACTCCCCGTCTGTGCCTTCTCATCTGCCGGACCGTGTGCACTTCGCTTCACCTCTGCAC
GTCGCATGGAGACCACCGTGAACGCCCACCGGAACCTGCCCAAGGTCTTGCATAAGAGGACTCTT
GGACTTTCAGCAATGTCATCGATATC
HPRT1 intron 1 DNA sequence that can be used as a stuffer
sequence in the pAV-CAG-Merlin-V2 construct. (SEQ ID NO: 6)
ACTAGTTACAGCTCTGGTAGCGGTAACCATGCGTATTTGACACACGAAGGAACTAGGGAAAAGGC
ATTAGGTCATTTCAAGCCGAAATTCACATGTGCTAGAATCCAGATTCCATGCTGACCGATGCCCC
AGGATATAGAAAATGAGAATCTGGTCCTTACCTTCAAGAACATTCTTAACCGTAATCAGCCTCTG
GTATCTTAGCTCCACCCTCACTGGTTTTTTCTTGTTTGTTGAACCGGCCAAGCTGCTGGCCTCCC
TCCTCAACCGTTCTGATCATGCTTGCTAAAATAGTCAAAACCCCGGCCAGTTAAATATGCTTTAG
CCTGCTTTATTATGATTATTTTTGTTGTTTTGGCAATGACCTGGTTACCTGTTGTTTCTCCCACT
AAAACTTTTTAAGGGCACTAGT
pAAV2_CAG_Merlin_Kan, a DNA vector. (SEQ ID NO: 7)
GTAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTGCTGGCCTTTTGCTCA
CATGTCCTGCAGGCAGCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTGGCCAACTCC
ATCACTAGGGGTTCCTGCGGCCGGTCGCGTCTAGTTATTAATGCATACTAGTTACAGCTCTGGTA
GCGGTAACCATGCGTATTTGACACACGAAGGAACTAGGGAAAAGGCATTAGGTCATTTCAAGCCG
AAATTCACATGTGCTAGAATCCAGATTCCATGCTGACCGATGCCCCAGGATATAGAAAATGAGAA
TCTGGTCCTTACCTTCAAGAACATTCTTAACCGTAATCAGCCTCTGGTATCTTAGCTCCACCCTC
ACTGGTTTTTTCTTGTTTGTTGAACCGGCCAAGCTGCTGGCCTCCCTCCTCAACCGTTCTGATCA
TGCTTGCTAAAATAGTCAAAACCCCGGCCAGTTAAATATGCTTTAGCCTGCTTTATTATGATTAT
TTTTGTTGTTTTGGCAATGACCTGGTTACCTGTTGTTTCTCCCACTAAAACTTTTTAAGGGCACT
AGTTACCATTGACGTCAATAATGACGTATGTTCCCATAGTAACGCCAATAGGGACTTTCCATTGA
CGTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCATGGTCGAG
GTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCG
GGGCGGGGCGGGGCGAGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCCCTATAAAAAGCGAAG
CGCGCGGCGGGCGGGAGTCGCTGCGACGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCCCTTC
TCCTCCGGGCTGTAATTAGCGCTTGGTTTAATGACGGCTTGTTTCTTTTCTGTGGCTGCGTGAAA
GCCTTGAGGGGCTCCGGGAGGGCCCTTTGTGCGGGGGGAGCGGCTCGGGGCTGTCCGCGGGGGGA
CGGCTGCCTTCGGGGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCAACGTGCTGGTT
ATTGTGCTGTCTCATCATTTTGGCAAAGAATTGGATCGGTACCAGCCACCATGGCCGGCGCGATT
GCTTCCCGGATGTCATTCTCCTCACTTAAAAGAAAACAGCCTAAGACTTTTACCGTGCGGATCGT
GACTATGGATGCAGAGATGGAATTCAACTGCGAGATGAAGTGGAAGGGAAAGGACCTCTTCGACC
TCGTCTGTCGCACCTTGGGACTGCGGGAAACCTGGTTCTTCGGACTGCAGTACACAATCAAGGAC
ACCGTGGCCTGGCTGAAAATGGACAAGAAGGTCCTGGACCACGACGTGTCCAAGGAAGAACCCGT
GACTTTCCACTTTCTGGCCAAGTTCTACCCGGAGAACGCTGAGGAAGAACTCGTGCAGGAGATCA
CTCAGCATCTGTTCTTTTTACAAGTCAAGAAGCAAATCCTAGATGAGAAGATCTACTGCCCGCCG
GAAGCGTCCGTGCTTCTGGCTTCCTACGCGGTGCAGGCCAAATATGGAGACTACGATCCCTCCGT
GCACAAGCGCGGGTTCCTGGCCCAAGAGGAACTGCTGCCTAAGCGCGTGATCAACCTGTACCAGA
TGACCCCCGAGATGTGGGAGGAACGAATTACGGCTTGGTACGCGGAGCATCGCGGCAGAGCACGC
GATGAAGCCGAGATGGAGTACCTGAAGATCGCGCAGGATCTTGAGATGTACGGGGTCAACTATTT
CGCCATCCGCAACAAGAAGGGCACCGAACTCCTGCTTGGAGTGGACGCACTCGGGCTCCACATCT
ACGACCCGGAAAACCGCCTGACTCCCAAGATCAGCTTCCCTTGGAACGAAATCAGAAACATTTCC
TACTCGGATAAGGAATTCACGATCAAGCCACTGGACAAAAAGATTGACGTGTTCAAGTTCAACTC
GTCGAAGCTGCGCGTGAACAAGCTGATACTGCAACTGTGCATTGGCAATCACGACCTGTTTATGC
GCCGGCGGAAGGCCGACTCATTGGAGGTCCAACAGATGAAGGCCCAGGCCCGCGAGGAAAAGGCT
CGCAAGCAGATGGAACGGCAGAGGCTGGCCAGAGAGAAGCAGATGCGGGAAGAGGCCGAACGCAC
CCGGGATGAACTGGAGCGCAGGCTGCTGCAGATGAAGGAAGAAGCAACCATGGCCAACGAAGCGC
TGATGCGGAGCGAAGAAACCGCGGACCTGTTGGCCGAAAAGGCTCAGATCACCGAGGAAGAAGCC
AAGCTCCTTGCGCAAAAGGCCGCCGAGGCCGAACAGGAGATGCAGAGAATCAAGGCCACCGCCAT
CAGAACCGAAGAAGAGAAGCGGCTCATGGAACAGAAAGTGTTGGAAGCCGAGGTGCTGGCGCTTA
AGATGGCAGAGGAGTCCGAGAGAAGGGCCAAAGAGGCAGACCAGCTGAAGCAGGATCTGCAGGAA
GCCCGGGAAGCCGAGCGGCGGGCGAAGCAGAAGCTCTTGGAAATCGCCACCAAACCGACTTACCC
ACCGATGAATCCTATTCCCGCCCCCCTCCCCCCTGACATTCCCTCCTTCAACCTCATCGGAGACT
CCCTGTCCTTCGACTTCAAAGACACCGATATGAAGCGGCTGTCTATGGAAATTGAAAAGGAAAAG
GTCGAGTACATGGAGAAGTCCAAGCATCTGCAAGAGCAACTCAACGAACTCAAAACTGAGATCGA
AGCTCTGAAGCTGAAGGAAAGAGAGACTGCCCTGGACATTCTCCATAACGAAAACTCGGACAGGG
GTGGTAGCAGCAAGCACAACACCATCAAGAAGCTGACTCTGCAATCGGCCAAGAGCCGCGTGGCA
TTCTTCGAGGAGCTGTAACTCGAGTAAATAACAGGCCTATTGATTGGAAAGTTTGTCAACGAATT
GTGGGTCTTTTGGGGTTTGCTGCCCCTTTTACGCAATGTGGATATCCTGCTTTAATGCCTTTATA
TGCATGTATACAAGCAAAACAGGCTTTTACTTTCTCGCCAACTTACAAGGCCTTTCTCAGTAAAC
AGTATATGACCCTTTACCCCGTTGCTCGGCAACGGCCTGGTCTGTGCCAAGTGTTTGCTGACGCA
ACCCCCACTGGTTGGGGCTTGGCCATAGGCCATCAGCGCATGCGTGGAACCTTTGTGTCTCCTCT
GCCGATCCATACTGCGGAACTCCTAGCCGCTTGTTTTGCTCGCAGCAGGTCTGGAGCAAACCTCA
TCGGGACCGACAATTCTGTCGTACTCTCCCGCAAGTATACATCGTTTCCATGGCTGCTAGGCTGT
GCTGCCAACTGGATCCTGCGCGGGACGTCCTTTGTTTACGTCCCGTCGGCGCTGAATCCCGCGGA
CGACCCCTCCCGGGGCCGCTTGGGGCTCTACCGCCCGCTTCTCCGTCTGCCGTACCGTCCGACCA
CGGGGCGCACCTCTCTTTACGCGGACTCCCCGTCTGTGCCTTCTCATCTGCCGGACCGTGTGCAC
TTCGCTTCACCTCTGCACGTCGCATGGAGACCACCGTGAACGCCCACCGGAACCTGCCCAAGGTC
TTGCATAAGAGGACTCTTGGACTTTCAGCAATGTCATCGATATCCGATCCACCGGATCTAGATAA
CTGATCATAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCACTGCATTCT
AGTTGTGGTTTGTCCAAACTCATCAATGTATCTTAACGCGGTAACCACGTGCGGACCCAACGGCC
GCAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCGC
AGCTGCCTGCAGGGGCGCCTGATGCGGTATTTTCTCCTTACGCATCTGTGCGGTATTTCACACCG
CATACGTCAAAGCAACCATAGTACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTTAGGGTTC
CGATTTAGTGCTTTACGGCACCTCGACCCCAAAAAACTTGATTTGGGTGATGGTTCACGTAGTGG
GCCATCGCCCTGATAGACGGTTTTTCGCCCTTTGACGTTGGAGTCCACGTTCTTTAATAGTGGAC
TCTTGTTCCAAACTGGAACAACACTCAACCCTATCTCGGGCTATTCTTTTGATTTATAAGGGATT
TTGCCGATTTCGGCCTATTGGTTAAAAAATGAGCTGATTTAACAAAAATTTAACGCGAATTTTAA
CAAAATATTAACGTTTACAATTTTATGGTGCACTCTCAGTACAATCTGCTCTGATGCCGCATAGT
TAAGCCAGCCCCGACACCCGCCAACACCCGCTGACGCGCCCTGACGGGCTTGTCTGCTCCCGGCA
TCCGCTTACAGACAAGCTGTGACCGTCTCCGGGAGCTGCATGTGTCAGAGGTTTTCACCGTCATC
ACCGAAACGCGCGAGACGAAAGGGCCTCGTGATACGCCTATTTTTATAGGTTAATGTCATGACGC
GGAACCCCTATTTGTTTATTTTTCTAAATACATTCAAATATGTATCCGCTCATGAGACAATAACC
CTGATAAATGCTTCAATAATATTGAAAAAGGAAGAGTATGAGCCATATTCAACGGGAAACGTCGA
GGCCGCGATTAAATTCCAACATGGATGCTGATTTATATGGGTATAAATGGGCTCGCGATAATGTC
GGGCAATCAGGTGCGACAATCTATCGCTTGTATGGGAAGCCCGATGCGCCAGAGTTGTTTCTGAA
ACATGGCAAAGGTAGCGTTGCCAATGATGTTACAGATGAGATGGTCAGACTAAACTGGCTGACGG
AATTTATGCCTCTTCCGACCATCAAGCATTTTATCCGTACTCCTGATGATGCATGGTTACTCACC
ACTGCGATCCCCGGAAAAACAGCATTCCAGGTATTAGAAGAATATCCTGATTCAGGTGAAAATAT
TGTTGATGCGCTGGCAGTGTTCCTGCGCCGGTTGCATTCGATTCCTGTTTGTAATTGTCCTTTTA
ACAGCGATCGCGTATTTCGTCTCGCTCAGGCGCAATCACGAATGAATAACGGTTTGGTTGATGCG
AGTGATTTTGATGACGAGCGTAATGGCTGGCCTGTTGAACAAGTCTGGAAAGAAATGCATAAACT
TTTGCCATTCTCACCGGATTCAGTCGTCACTCATGGTGATTTCTCACTTGATAACCTTATTTTTG
ACGAGGGGAAATTAATAGGTTGTATTGATGTTGGACGAGTCGGAATCGCAGACCGATACCAGGAT
CTTGCCATCCTATGGAACTGCCTCGGTGAGTTTTCTCCTTCATTACAGAAACGGCTTTTTCAAAA
ATATGGTATTGATAATCCTGATATGAATAAATTGCAGTTTCATTTGATGCTCGATGAGTTTTTCT
AATCATGACCAAAATCCCTTAACGTGAGTTTTCGTTCCACTGAGCGTCAGACCCCGTAGAAAAGA
TCAAAGGATCTTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGAAGGTAACTGG
CTTCAGCAGAGCGCAGATACCAAATACTGTTCTTCTAGTGTAGCCGTAGTTAGGCCACCACTTCA
AGAACTCT
MERLIN ISOFORM 2 protein (SEQ ID NO: 8):
MAGAIASRMSFSSLKRKQPKTFTVRIVTMDAEMEFNCEMKWKGKDLFDLVCRTLGLRETWFFGLQ
YTIKDTVAWLKMDKKVLDHDVSKEEPVTFHFLAKFYPENAEEELVQEITQHLFFLQVKKQILDEK
IYCPPEASVLLASYAVQAKYGDYDPSVHKRGFLAQEELLPKRVINLYQMTPEMWEERITAWYAEH
RGRARDEAEMEYLKIAQDLEMYGVNYFAIRNKKGTELLLGVDALGLHIYDPENRLTPKISFPWNE
IRNISYSDKEFTIKPLDKKIDVFKFNSSKLRVNKLILQLCIGNHDLFMRRRKADSLEVQQMKAQA
REEKARKQMERQRLAREKQMREEAERTRDELERRLLQMKEEATMANEALMRSEETADLLAEKAQI
TEEEAKLLAQKAAEAEQEMQRIKATAIRTEEEKRLMEQKVLEAEVLALKMAEESERRAKEADQLK
QDLQEAREAERRAKQKLLEIATKPTYPPMNPIPAPLPPDIPSFNLIGDSLSFDFKDTDMKRLSME
IEKEKVEYMEKSKHLQEQLNELKTEIEALKLKERETALDILHNENSDRGGSSKHNTIKKPQAQGR
RPICI
MERLIN ISOFORM 2 coding region (SEQ ID NO: 9):
ATGGCCGGGGCCATCGCTTCCCGCATGAGCTTCAGCTCTCTCAAGAGGAAGCAACCCAAGACGTT
CACCGTGAGGATCGTCACCATGGACGCCGAGATGGAGTTCAATTGCGAGATGAAGTGGAAAGGGA
AGGACCTCTTTGATTTGGTGTGCCGGACTCTGGGGCTCCGAGAAACCTGGTTCTTTGGACTGCAG
TACACAATCAAGGACACAGTGGCCTGGCTCAAAATGGACAAGAAGGTACTGGATCATGATGTTTC
AAAGGAAGAACCAGTCACCTTTCACTTCTTGGCCAAATTTTATCCTGAGAATGCTGAAGAGGAGC
TGGTTCAGGAGATCACACAACATTTATTCTTCTTACAGGTAAAGAAGCAGATTTTAGATGAAAAG
ATCTACTGCCCTCCTGAGGCTTCTGTGCTCCTGGCTTCTTACGCCGTCCAGGCCAAGTATGGTGA
CTACGACCCCAGTGTTCACAAGCGGGGATTTTTGGCCCAAGAGGAATTGCTTCCAAAAAGGGTAA
TAAATCTGTATCAGATGACTCCGGAAATGTGGGAGGAGAGAATTACTGCTTGGTACGCAGAGCAC
CGAGGCCGAGCCAGGGATGAAGCTGAAATGGAATATCTGAAGATAGCTCAGGACCTGGAGATGTA
CGGTGTGAACTACTTTGCAATCCGGAATAAAAAGGGCACAGAGCTGCTGCTTGGAGTGGATGCCC
TGGGGCTTCACATTTATGACCCTGAGAACAGACTGACCCCCAAGATCTCCTTCCCGTGGAATGAA
ATCCGAAACATCTCGTACAGTGACAAGGAGTTTACTATTAAACCACTGGATAAGAAAATTGATGT
CTTCAAGTTTAACTCCTCAAAGCTTCGTGTTAATAAGCTGATTCTCCAGCTATGTATCGGGAACC
ATGATCTATTTATGAGGAGAAGGAAAGCCGATTCTTTGGAAGTTCAGCAGATGAAAGCCCAGGCC
AGGGAGGAGAAGGCTAGAAAGCAGATGGAGCGGCAGCGCCTCGCTCGAGAGAAGCAGATGAGGGA
GGAGGCTGAACGCACGAGGGATGAGTTGGAGAGGAGGCTGCTGCAGATGAAAGAAGAAGCAACAA
TGGCCAACGAAGCACTGATGCGGTCTGAGGAGACAGCTGACCTGTTGGCTGAAAAGGCCCAGATC
ACCGAGGAGGAGGCAAAACTTCTGGCCCAGAAGGCCGCAGAGGCTGAGCAGGAAATGCAGCGCAT
CAAGGCCACAGCGATTCGCACGGAGGAGGAGAAGCGCCTGATGGAGCAGAAGGTGCTGGAAGCCG
AGGTGCTGGCACTGAAGATGGCTGAGGAGTCAGAGAGGAGGGCCAAAGAGGCAGATCAGCTGAAG
CAGGACCTGCAGGAAGCACGCGAGGCGGAGCGAAGAGCCAAGCAGAAGCTCCTGGAGATTGCCAC
CAAGCCCACGTACCCGCCCATGAACCCAATTCCAGCACCGTTGCCTCCTGACATACCAAGCTTCA
ACCTCATTGGTGACAGCCTGTCTTTCGACTTCAAAGATACTGACATGAAGCGGCTTTCCATGGAG
ATAGAGAAAGAAAAAGTGGAATACATGGAAAAGAGCAAGCATCTGCAGGAGCAGCTCAATGAACT
CAAGACAGAAATCGAGGCCTTGAAACTGAAAGAGAGGGAGACAGCTCTGGATATTCTGCACAATG
AGAACTCCGACAGGGGTGGCAGCAGCAAGCACAATACCATTAAAAAGCCTCAAGCCCAAGGCAGA
AGACCTATCTGCATT

Although the embodiments have been described with respect to various preferred embodiments, it is not intended to be limited thereto, but rather those skilled in the art will recognize that variations and modifications may be made therein which are within the spirit of the embodiments and the scope of the appended claims.

### EXAMPLES

### Example 1. AAV Design Encoding for Merlin Isoform 1

An AAV plasmid to produce an AAV vector encoding for Merlin Isoform 1 was designed. The transgene was subcloned into pAV-ACG (Vigene Biosciences) at the KpnI and XhoI sites. The entire AAV genome (excluding the ITRs) was sequence confirmed.

The sequence of Merlin is as follows:

(SEQ ID NO: 1)
MAGAIASRMSFSSLKRKQPKTFTVRIVTMDAEMEFNCEMKWKGKDLFDLV
CRTLGLRETWFFGLQYTIKDTVAWLKMDKKVLDHDVSKEEPVTFHFLAKF
YPENAEEELVQEITQHLEFLQVKKQILDEKIYCPPEASVLLASYAVQAKY
GDYDPSVHKRGFLAQEELLPKRVINLYQMTPEMWEERITAWYAEHRGRAR
DEAEMEYLKIAQDLEMYGVNYFAIRNKKGTELLLGVDALGLHIYDPENRL
TPKISFPWNEIRNISYSDKEFTIKPLDKKIDVFKFNSSKLRVNKLILQLC
IGNHDLFMRRRKADSLEVQQMKAQAREEKARKQMERQRLAREKQMREEAE
RTRDELERRLLQMKEEATMANEALMRSEETADLLAEKAQITEEEAKLLAQ
KAAEAEQEMQRIKATAIRTEEEKRLMEQKVLEAEVLALKMAEESERRAKE
ADQLKQDLQEAREAERRAKQKLLEIATKPTYPPMNPIPAPLPPDIPSFNL
IGDSLSFDFKDTDMKRLSMEIEKEKVEYMEKSKHLQEQLNELKTEIEALK
LKERETALDILHNENSDRGGSSKHNTIKKLTLQSAKSRVAFFEEL

The nucleotide sequence provided in the vector is simply illustrative and due to the degeneracy of the genetic code other nucleotide sequences can be used to encode for the Merlin protein. In the present example, the following nucleotide sequence can be used to encode for the Merlin protein (the stop codon is not shown)

(SEQ ID NO: 2)
ATGGCCGGCGCGATTGCTTCCCGGATGTCATTCTCCTCACTTAAAAGAAA
ACAGCCTAAGACTTTTACCGTGCGGATCGTGACTATGGATGCAGAGATGG
AATTCAACTGCGAGATGAAGTGGAAGGGAAAGGACCTCTTCGACCTCGTC
TGTCGCACCTTGGGACTGCGGGAAACCTGGTTCTTCGGACTGCAGTACAC
AATCAAGGACACCGTGGCCTGGCTGAAAATGGACAAGAAGGTCCTGGACC
ACGACGTGTCCAAGGAAGAACCCGTGACTTTCCACTTTCTGGCCAAGTTC
TACCCGGAGAACGCTGAGGAAGAACTCGTGCAGGAGATCACTCAGCATCT
GTTCTTTTTACAAGTCAAGAAGCAAATCCTAGATGAGAAGATCTACTGCC
CGCCGGAAGCGTCCGTGCTTCTGGCTTCCTACGCGGTGCAGGCCAAATAT
GGAGACTACGATCCCTCCGTGCACAAGCGCGGGTTCCTGGCCCAAGAGGA
ACTGCTGCCTAAGCGCGTGATCAACCTGTACCAGATGACCCCCGAGATGT
GGGAGGAACGAATTACGGCTTGGTACGCGGAGCATCGCGGCAGAGCACGC
GATGAAGCCGAGATGGAGTACCTGAAGATCGCGCAGGATCTTGAGATGTA
CGGGGTCAACTATTTCGCCATCCGCAACAAGAAGGGCACCGAACTCCTGC
TTGGAGTGGACGCACTCGGGCTCCACATCTACGACCCGGAAAACCGCCTG
ACTCCCAAGATCAGCTTCCCTTGGAACGAAATCAGAAACATTTCCTACTC
GGATAAGGAATTCACGATCAAGCCACTGGACAAAAAGATTGACGTGTTCA
AGTTCAACTCGTCGAAGCTGCGCGTGAACAAGCTGATACTGCAACTGTGC
ATTGGCAATCACGACCTGTTTATGCGCCGGCGGAAGGCCGACTCATTGGA
GGTCCAACAGATGAAGGCCCAGGCCCGCGAGGAAAAGGCTCGCAAGCAGA
TGGAACGGCAGAGGCTGGCCAGAGAGAAGCAGATGCGGGAAGAGGCCGAA
CGCACCCGGGATGAACTGGAGCGCAGGCTGCTGCAGATGAAGGAAGAAGC
AACCATGGCCAACGAAGCGCTGATGCGGAGCGAAGAAACCGCGGACCTGT
TGGCCGAAAAGGCTCAGATCACCGAGGAAGAAGCCAAGCTCCTTGCGCAA
AAGGCCGCCGAGGCCGAACAGGAGATGCAGAGAATCAAGGCCACCGCCAT
CAGAACCGAAGAAGAGAAGCGGCTCATGGAACAGAAAGTGTTGGAAGCCG
AGGTGCTGGCGCTTAAGATGGCAGAGGAGTCCGAGAGAAGGGCCAAAGAG
GCAGACCAGCTGAAGCAGGATCTGCAGGAAGCCCGGGAAGCCGAGCGGCG
GGCGAAGCAGAAGCTCTTGGAAATCGCCACCAAACCGACTTACCCACCGA
TGAATCCTATTCCCGCCCCCCTCCCCCCTGACATTCCCTCCTTCAACCTC
ATCGGAGACTCCCTGTCCTTCGACTTCAAAGACACCGATATGAAGCGGCT
GTCTATGGAAATTGAAAAGGAAAAGGTCGAGTACATGGAGAAGTCCAAGC
ATCTGCAAGAGCAACTCAACGAACTCAAAACTGAGATCGAAGCTCTGAAG
CTGAAGGAAAGAGAGACTGCCCTGGACATTCTCCATAACGAAAACTCGGA
CAGGGGTGGTAGCAGCAAGCACAACACCATCAAGAAGCTGACTCTGCAAT
CGGCCAAGAGCCGCGTGGCATTCTTCGAGGAGCTG

Additionally, although the present example, and those below, illustrate the use of Isoform 1 of Merlin, Isoform 2 of Merlin could also be used in its place.

### Example 2: Vector can Express Merlin Protein

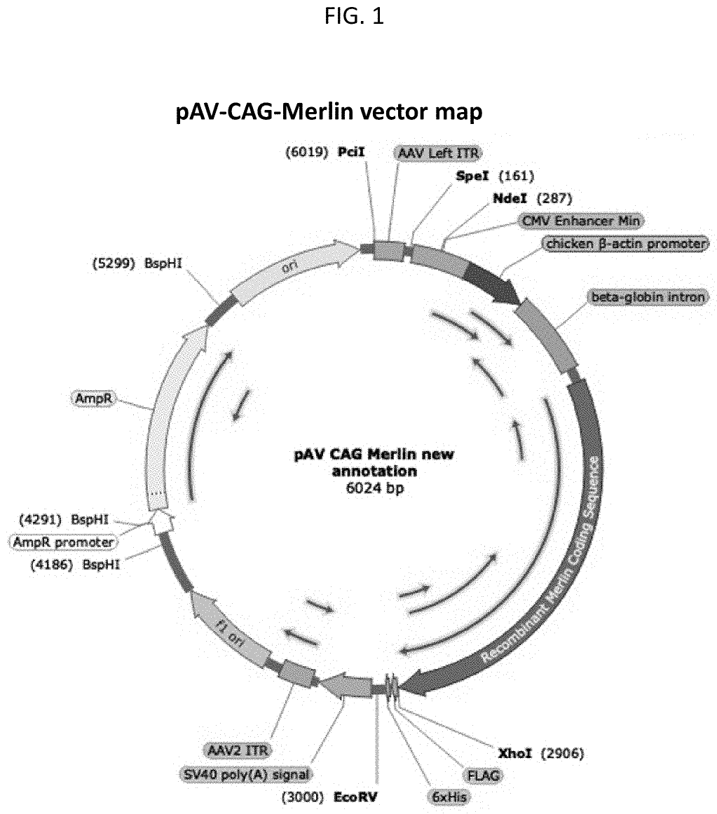

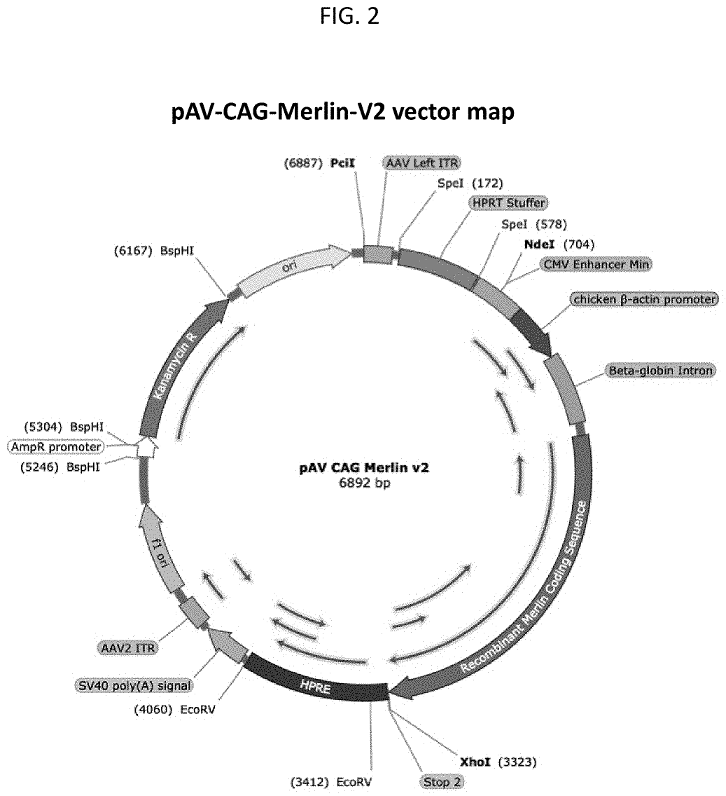

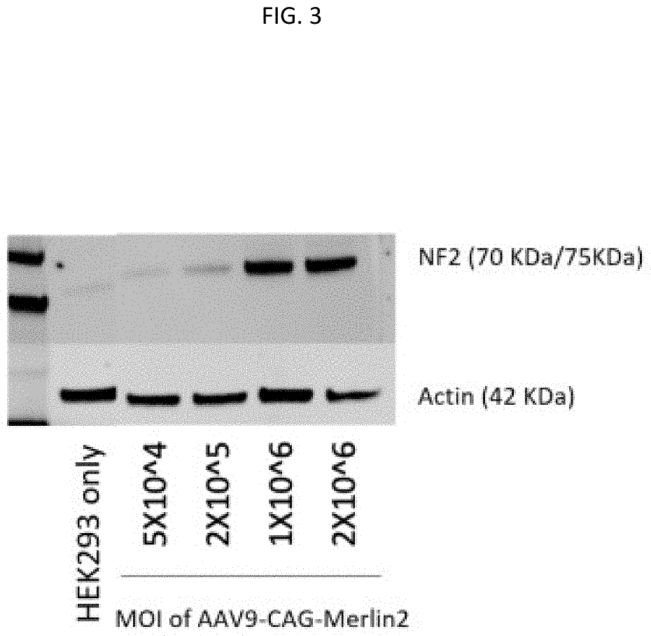

An AAV9 vector was used to transduce HEK293 cells, the vector can be based off a plasmid s shown in FIG. 1 or FIG. 2. The AAV9 vector comprised a transgene encoding for Merlin protein. The cells were analyzed for expression of Merlin protein and was detected. These results are illustrated in FIG. 3, which shows a Western Blot detecting the expression of the protein.

### Example 3. Viral Delivery of Merlin to Treat NF2

An adeno-associated virus (AAV) encoding normal (wild-type) NF2 (Merlin) is administered to the central nervous system (CNS) of a patient suffering from NF2. The AAV is administered by intrathecal injection, but could also be administered by multiple intertumoral injections, intraventricularly, or intravenously. The expression of Merlin in the CNS halts tumor progression and/or leads to tumor regression. The AAV comprises the AAV capsid, and the NF2 transgene operatively connected to a constitutive promoter. The AAV is based on AAV-DJ (Grimm D, et al. (2008) In vitro and in vivo gene therapy vector evolution via multispecies interbreeding and retargeting of adeno-associated viruses. J Virol. 82:5887-911.) or AAV-9 (Gao G. et al. (2004) Clades of Adeno-associated viruses are widely disseminated in human tissues. J Virol. 78:6381-8; Hocquemiller, M. et al. (2016) Adeno-Associated Virus-Based Gene Therapy for CNS Diseases. Hum Gene Ther. 7:478-496). The constitutive promoter is CAG (Fitzsimons, et al. (2002) Promoters and regulatory elements that improve adeno-associated virus transgene expression in the brain. Methods 28:227-36.). Without being bound to any particular theory, the use of this promoter enables the transgene to be constitutively expressed in most cells. However, a tissue specific promoter can be used. The NF2 transgene is a human sequence-optimized neurofibromatosis 2 (NF2) gene, which can also be referred to as “h-soNF2.” The isoform of NF2 being used will be the longest (595 amino acids), but other isoforms, or active fragments thereof, could be used in its place.

### Example 4. Expression of NF2 Reduces Spinal Schwannomas in NF2 Transgenic Mice and Meningioma Xenografts in Nude (SCID) Mice

The AAV of Example 1 is delivered intrathecally to adult transgenic NF2 mice with spinal schwannomas or nude mice with meningioma xenografts. The mice are monitored, and then upon sacrifice tumor cells and normal Schwann and arachnoid cells are harvested. The presence of full length human merlin is confirmed via western blot. Volumetric measurements of tumors are made and the tumor burden is found to be reduced.

### Example 5. AAV-NF2 is Functional in Cell Culture Models of Neurofibromatosis Type 2

Initially the AAV of Example 1, is used to deliver the merlin gene to cells and the gene is functional and produces the merlin protein. Human merlin production from the AAV-delivered transgene is assessed by western blot method using human merlin-specific antibodies. The cell line can be, for example, a RT4 Rat Schwannoma Cell line. Colony formation assays are performed as well and the AAV-NF2 is found to reduce colony formation in a soft agar assay.

### Example 6. pAV-CAG-Merlin-V2 is an Improved AAV Gene Therapy Plasmid

pAV-CAG-Merlin-V2 was generated by modifying sequences elements in the original Merlin construct (pAV-CAG-Merlin) as shown in FIG. 1 and FIG. 2. The cloning strategy is based on the nucleotide position in the original Merlin Construct. pAV-CAG-Merlin-V2 is an recombinant adeno-associated virus (AAV) DNA plasmid with 6892 base pair (bp) in size (FIG. 2), which contains inverted terminal repeat (ITR) sequences derived from AAV serotype 2 (AAV2) and a transgene cassette expressing Merlin protein. In pAV-CAG-Merlin-V2, the Left-ITR is modified to include a stuffer sequence, which can be used to confer optimal AAV packaging efficiency. A HPRE sequence is inserted downstream of sequences encoding the Merlin protein in order to facilitate transgene expression. The vector was modified to include a Kanamycin resistance gene for ease of manufacture of the DNA product used in recombinant AAV production.

The expression cassette is comprised of a CAG promoter coupled to sequence encoding a codon optimized Merlin protein followed by a post-transcriptional regulatory element of hepatitis B virus (HPRE) and then a transcription termination SV40 Polyadenylation (SV40-polyA) sequence. Without being bound to any particular theory, the HPRE element is a cis-acting sequence that facilitates the cytoplasmic localization of intronless transcripts and contributes to higher gene expression. In addition, a 400 bp stuffer sequence derived from the intron 1 region of the Hypoxanthine Phospho-ribosyl-transferase 1 (HPRT1) gene is inserted downstream of the left ITR and upstream of the CAG promoter to extend AAV package size to 4487 bp. A Kanamycin resistance gene (KanR) is included in the plasmid backbone as a selection marker.

To produce a plasmid to increase AAV packaging efficiency, the left ITR sequence can be modified. The Left-ITR can comprise:

(SEQ ID NO: 4)
ACATGTCCTGCAGGCAGCTGCGCGCTCGCTCGCTCACTGAGGCCGCCCGG
GCAAAGCCCGGGCGTCGGGCGACCTTTGGTCGCCCGGCCTCAGTGAGCGA
GCGAGCGCGCAGAGAGGGAGTGGCCAACTCCATCACTAGGGGTTCCTGCG
GCCGGTCGCGTCTAGTTATTAATGCATACTAGTTACCATTGACGTCAATA
ATGACGTATGTTCCCATAGTAACGCCAATAGGGACTTTCCATTGACGTCA
ATGGGTGGACTATTTACGGTAAACTGCCCACTTGGCAGTACATCAAGTGT
ATCATATG

A vector was created that removes any artificial tags, such as a histidine tag or FLAG tag. Transgenes carried by a gene therapy vector are mostly devoid of intron sequences. Intronless transcripts can be less stable and prone to accumulation inside the nucleus. Posttranscriptional regulatory elements (PREs) of Hepatitis B virus (HPRE) can increase accumulation of the cytoplasmic mRNA of an intronless gene by promoting mRNA exportation from the nucleus to the cytoplasm, enhancing 3′ end processing and stability. Therefore, a HPRE element is added to the recombinant Merlin transcript to improve transgene mRNA stability and transgene expression. The HPRE fragment is synthesized and inserted between XhoI site (2906) and EcoRV site (3000). The Posttranscriptional regulatory elements (PREs) of Hepatitis B virus (HPRE) can be as follows:

(SEQ ID NO: 5)
CTCGAGTAAATAACAGGCCTATTGATTGGAAAGTTTGTCAACGAATTGTG
GGTCTTTTGGGGTTTGCTGCCCCTTTTACGCAATGTGGATATCCTGCTTT
AATGCCTTTATATGCATGTATACAAGCAAAACAGGCTTTTACTTTCTCGC
CAACTTACAAGGCCTTTCTCAGTAAACAGTATATGACCCTTTACCCCGTT
GCTCGGCAACGGCCTGGTCTGTGCCAAGTGTTTGCTGACGCAACCCCCAC
TGGTTGGGGCTTGGCCATAGGCCATCAGCGCATGCGTGGAACCTTTGTGT
CTCCTCTGCCGATCCATACTGCGGAACTCCTAGCCGCTTGTTTTGCTCGC
AGCAGGTCTGGAGCAAACCTCATCGGGACCGACAATTCTGTCGTACTCTC
CCGCAAGTATACATCGTTTCCATGGCTGCTAGGCTGTGCTGCCAACTGGA
TCCTGCGCGGGACGTCCTTTGTTTACGTCCCGTCGGCGCTGAATCCCGCG
GACGACCCCTCCCGGGGCCGCTTGGGGCTCTACCGCCCGCTTCTCCGTCT
GCCGTACCGTCCGACCACGGGGCGCACCTCTCTTTACGCGGACTCCCCGT
CTGTGCCTTCTCATCTGCCGGACCGTGTGCACTTCGCTTCACCTCTGCAC
GTCGCATGGAGACCACCGTGAACGCCCACCGGAACCTGCCCAAGGTCTTG
CATAAGAGGACTCTTGGACTTTCAGCAATGTCATCGATATC

A stuffer sequence is introduced at the 5′ end of the construct in front of the CAG promoter. The optimal packaging size of an AAV vector is 4.1˜4.9 kb (Grieger et al. 2005). The packaging size of the original Merlin construct was just 3.5 kb. To increase the AAV vector packaging size, a 400 bp stuffer sequence derived from the intron 1 region of the HPRT1 gene is inserted downstream of the left ITR sequence and upstream of the CAG promoter sequence. With this sequence addition, the AAV is increased to facilitate efficient packaging into the AAV capsid. A HPRT1 intron 1 sequence is used as a stuffer sequence, but other stuffer sequences can be used: The stuffer sequence can be as follows:

(SEQ ID NO: 6)
ACTAGTTACAGCTCTGGTAGCGGTAACCATGCGTATTTGACACACGAAGG
AACTAGGGAAAAGGCATTAGGTCATTTCAAGCCGAAATTCACATGTGCTA
GAATCCAGATTCCATGCTGACCGATGCCCCAGGATATAGAAAATGAGAAT
CTGGTCCTTACCTTCAAGAACATTCTTAACCGTAATCAGCCTCTGGTATC
TTAGCTCCACCCTCACTGGTTTTTTCTTGTTTGTTGAACCGGCCAAGCTG
CTGGCCTCCCTCCTCAACCGTTCTGATCATGCTTGCTAAAATAGTCAAAA
CCCCGGCCAGTTAAATATGCTTTAGCCTGCTTTATTATGATTATTTTTGT
TGTTTTGGCAATGACCTGGTTACCTGTTGTTTCTCCCACTAAAACTTTTT
AAGGGCACTAGT

### Example 7. Merlin Expressed in Cerebellum from an AAV Vector in Cynomolgus Macaques

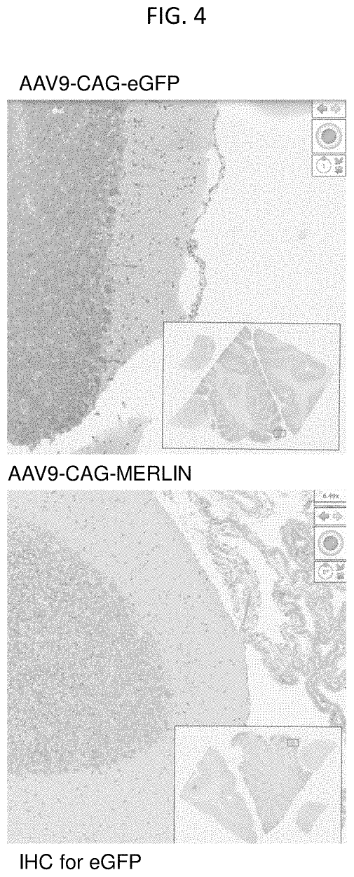

A study was performed to evaluate safety and biodistribution of AAV9-CAG-Merlin-v2 and AAV9-CAG-eGFP for the treatment of Neurofibromatosis type 2 (NF2), when given intracisternal magna (ICM) to cynomolgus monkeys. An additional objective was to confirm transgene delivery and expression from each of the vectors in the Schwann, arachnoid, and ependymal cells in the regions of interest. Sections of each collected tissue from were analyzed for green fluorescent protein (GFP; Rabbit anti-GFP) (via immunostaining). Scans of each tissue sample were prepared for immunostaining of GFP, and immunofluorescence. FIG. 4 shows immunohistochemistry images of biodistribution of eGFP in the cerebellum of in cynomolgus macaques dosed with AAV9-CAG-eGFP by ICM administration. These data use surrogate biomarker biodistribution demonstrate that AAV9 vectors provided by the ICM route of administration can express a protein in proper tissues and perhaps provide evidence that AAV9 vectors expressing the merlin protein can restore normal merlin function. The eGFP expression was used as a surrogate marker for Merlin expression since the animal expresses native Merlin protein, which was not distinguishable from the merlin expressed from the AAV9 vector.

### Example 8. In NF2 Knockout Mice Administered with AAV9-CAG-Merlin v2 there was a Reduction in Density of Schwann Cells Vs Mice Treated with AAV9-CAG-GFP

Postn-Cre;Nf2^{flox/flox} mice were injected into the cisternal magna (ICM) with 5 μL or 10 μL of test article (AAV9-CAG-Merlin or AAV9-CAG-Merlin-v2) or a negative control vector (AAV9-CAG-GFP). In the Postn-Cre;Nf2^{flox/flox} mouse model, SC hyperplasia is observed in the Dorsal Root Ganglion (DRG) of Postn-Cre;Nf2^{flox/flox} mice, similar to that observed in humans with NF2. At 6 months of age Postn-Cre;Nf2^{flox/flox} mice have significantly increased SC numbers (SC hyperplasia and schwannoma tumorlets) in DRGs compared to control mice. Periostin-Cre:NF2^{flox/flox} mice were bred with NF2^{flox/flox} mice.

Study Design:

| | | | | | | Average | Age at | Age at |
|---|---|---|---|---|---|---|---|---|
| | | | Route of | | Dose | Animal | Dosing | Sacrifice |
| Name of Virus | Group | # Mice | Admin | Lot Number | Level | Weight (kg) | (mo) | (mo) |
| AAV-9-CAG-GFP | 1a | 4 | ICM | Stanford 5796 | Low | 0.01 | 1 | 6 |
| AAV-9-CAG-Merlin | 2a | 2 | ICM | Stanford 5795 | Low | 0.01 | 1 | 6 |
| AAV-9-CAG-GFP | 1b | 4 | ICM | Stanford 6405 | High | 0.01 | 1 | 6 |
| AAV-9-CAG-Merlin v2 | 2b | 5 | ICM | Stanford 6404 | High | 0.01 | 1 | 6 |
| AAV-9-CAG-GFP | 3 | 1 | ICM | Stanford 5796 | Low | 0.01 | 1 | 2 |

Group 1a: 4 × 1 month old pups injected with AAV9-CAG-GFP (low dose control, 5.6E12 vg/kg)
Group 1b: 4 × 1 month old pups injected with AAV9-CAG-GFP (high dose control, 4E13 vg/kg)
Group 2a: 10 × 1 month old pups injected with AAV9-CAG-Merlin v1 (low dose, 5.6E12 vg/kg)
Group 2b: 10 × 1 month old pups injected with AAV9-CAG-Merlin v2 (high dose 7E13 vg/kg)
Group 3: 5 × 1 month old pups injected with AAV-GFP (low dose control, 5.6E12 vg/kg) sacrificed at 4 weeks post injection to analyze tissue distribution.

Post dosing mice were observed for 5 months (groups 1a, 1b, 2a, 2b), or 4 weeks (group 3). Two Group 1b mice (AAV9-CAG-GFP) were sacrificed after 22 days, and 50 days respectively due to excessive weight loss. Following the post dosing observation period, mice were sacrificed for further analysis. In order to assess anti-proliferative activity of AAV9-CAG-Merlin, counts of Schwann cell (SC) in Dorsal Root Ganglion (DRG) histological sections were measured by the number of SC nuclei/m² and statistical analysis of values for AAV9-CAG-Merlin injected mice vs. AAV9-CAG-GFP injected controls. Immunohistochemistry with GFP antibodies were used to analyze AAV-GFP expression and tissue distribution (Group 3) and persistence of AAV-GFP expression at 6 months (Group 2).

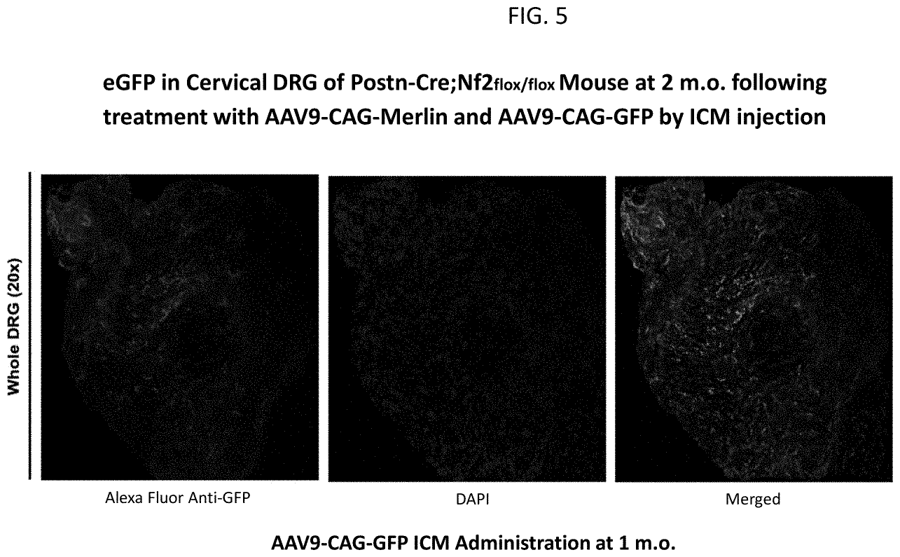

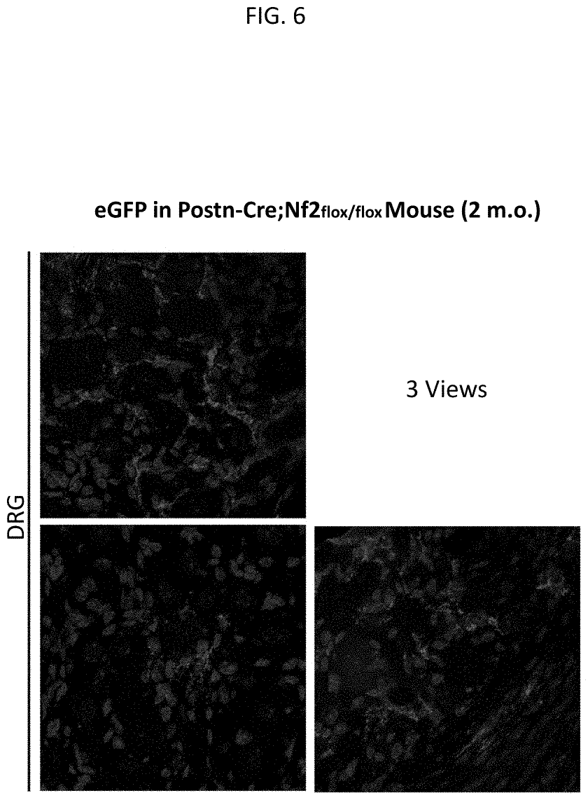

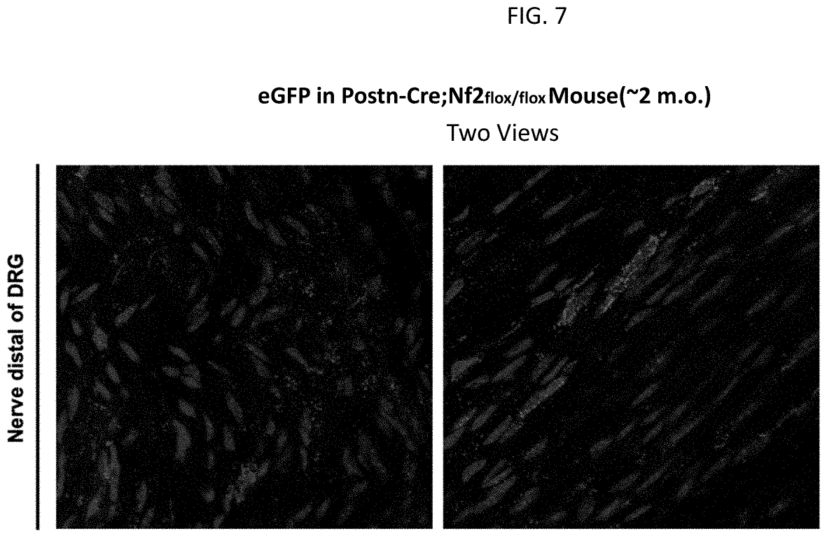

As a proxy for biodistribution of the AAV9-CAG-merlin constructs, an AAV9-CAG-eGFP construct, in which the Merlin transgene is replaced with a surrogate biomarker protein, was developed. It was expected that eGFP expression from this construct would correlate with the expected biodistribution of similar tissues transduced with the Merlin constructs. Significant biodistribution of eGFP is observed in DRG in Postn-Cre;Nf2^{flox/flox} mice injected with AAV9-CAG-GFP, as shown in the enhanced immunofluorescent micrographs in FIG. 5. Enhanced immunofluorescence analyses demonstrated the presence of eGFP expression in the cells surrounding axonal cells (Schwan cells) in a mouse administered with a low dose (group 3; 5.6 E12 vg/kg) of AAV9-CAG-GFP via ICM administration and was sacrificed 4 weeks later (FIG. 6). The nerve distal to the DRG also showed expression of eGFP in Schwann cells (FIG. 7). A section the full width of a DRG demonstrated distributed expression of eGFP across the full area of the DRG.

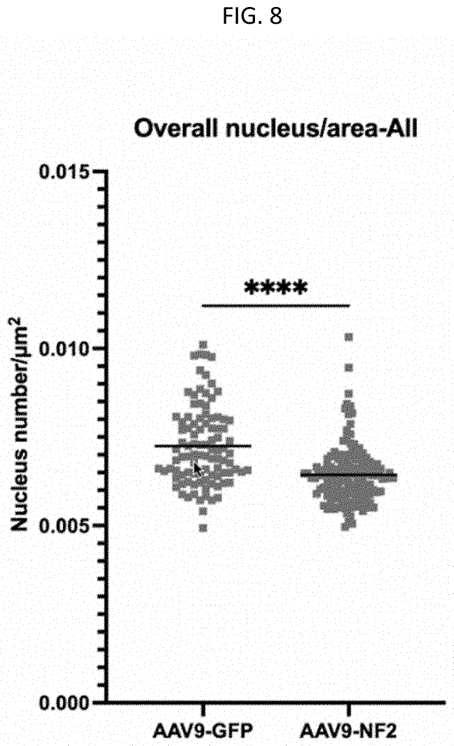

In order to assess anti-proliferative activity of AAV9-CAG-Merlin and AAV9-CAG-Merlin-v2, the number of nuclei per square micron of tissue were counted in AAV9-CAG-Merlin and AAV9-CAG-Merlin-v2 treated animals and compared against mice dosed with the negative control of AAV9-CAG-eGFP. A statistically significant reduction in number of SC nuclei/m² was observed in the high dose arm of AAV9-CAG-Merlin-v2 as compared to similar mice administered with an equivalent dose of AAV9-CAG-GFP. A small reduction (not statistically significant) in #SC nuclei/m² was observed in the low dose arm of AAV9-CAG-Merlin v2 as compared with AAV9-CAG-GFP. FIG. 8 shows significant reduction in the density of Schwann cell (SC) nuclei (nuclei/m²) in cross-sections of DRG from 6 month old Postn-Cre;Nf2^{flox/flox} mice 5 months post AAV9-CAG-Merlin administration compared to AAV9-CAG-GFP injected controls.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the compositions, methods, and the like have been described in conjunction with various embodiments and examples, it is not intended that they be limited to such embodiments or examples. On the contrary, the present disclosure encompasses various alternatives, modifications, and equivalents, as will be appreciated by those of skill in the art.

While the compositions and methods have been shown and described with reference to specific illustrative non-limiting embodiments, it should be understood that various changes in form and detail may be made without departing from the spirit and scope of the present disclosure. Therefore, all embodiments that come within the scope and spirit of the present disclosure, and equivalents thereto, are intended to be claimed. The claims, descriptions and diagrams of the compositions, methods, systems, and assays of the present disclosure should not be read as limited to the described order of elements unless stated to that effect.

1. An adeno-associated virus (AAV) comprising an AAV capsid protein and a transgene encoding a full-length Merlin protein or one, or more, active fragments thereof.

2. The AAV of claim 1, wherein the transgene is within AAV inverted terminal repeats, wherein the transgene is operably linked to regulatory sequences which direct expression of the heterologous gene in a host cell, and wherein the regulatory sequences comprise a constitutive or tissue specific promoter.

3-5. (canceled)

6. The AAV of claim 2, wherein the promoter is a CAG promoter, a CMV promoter, CBA promoter, or an SV40 promoter.

7. The AAV of claim 1, wherein the capsid protein is an AAV9 capsid protein.

8. (canceled)

9. The AAV of claim 7, wherein the AAV9 capsid comprises an amino acid sequence that is at least 80%, 85%, 90%, 95%, 96%, 97%, 98%, or 99% identical to amino acid residues 203 to 736 of SEQ ID NO: 3.

10. The AAV of claim 1, wherein the Merlin protein encoded by the transgene comprises a sequence that is at least 90%, 95%, 96%, 97%, 98%, or 99% identical to an amino acid sequence of SEQ ID NO: 1 or SEQ ID NO: 8.

11. The AAV of claim 1, wherein the Merlin protein encoded by the transgene comprises a sequence of SEQ ID NO: 1.

12-13. (canceled)

14. The AAV of claim 2, wherein the AAV inverted terminal repeats are AAV-2 inverted terminal repeats.

15. (canceled)

16. The AAV of claim 1, wherein the AAV comprises a nucleic acid molecule stuffer sequence upstream of the transgene and downstream of the 5′ AAV ITR.

17. (canceled)

18. The AAV of claim 1, wherein the AAV comprises a nucleotide intron sequence that is downstream of the transgene and upstream of the 3′ AAV ITR.

19. The AAV of claim 18, wherein the intron sequence is a HPRE sequence.

20-21. (canceled)

22. A pharmaceutical composition comprising the AAV of claim 1 and a pharmaceutically acceptable carrier.

23. A method of delivering a Merlin protein to a cell, the method comprising the step of contacting the cell with the AAV of claim 1.

24. A method of treating a subject with NF2, the method comprising administering to the subject with NF2 the AAV claim 1.

25. (canceled)

26. A method of inhibiting the growth of a schwannoma, a meningioma, or an ependymoma in a subject, the method comprising administering to the subject the AAV of claim 1.

27-31. (canceled)

32. A method of treating a subject with a merlin protein deficiency disorder, the method comprising administering to the subject the AAV of claim 1.

33. The method of claim 32, wherein the disorder is neurofibromatosis type 2, schwannomatosis, schwannomas, a cancer, a leukemia, a lymphoma, a chronic myeloproliferative disorder, langerhans cell histiocytosis, multiple myeloma/plasma cell neoplasm, a myelodysplastic syndrome, a myelodysplastic/myeloproliferative neoplasm, an ovarian cancer, breast cancer, invasive breast carcinoma, or a neurocutaneous disorder, mesothelioma, peritoneal mesothelioma, skin squamous cell carcinoma, cancer of the urinary tract, thyroid cancer, stomach cancer, schwannoma, renal cell carcinoma, cancer of the pituitary, ovarian cancer, meningioma, melanoma, lung cancer, liver cancer, large intestine cancer, hepatocellular carcinoma, acute myelogenous leukemia (AML), aerodigestive tract cancer, bladder cancer, bone cancer, colorectal carcinoma, ependymoma, colorectal carcinoma, endometrium, a glioma, cataracts, retinal detachment, damage to the nerves of the eye, papilledema, ocular migraine, retinitis pigmentosa (RP), combined hamartoma of the retina and RPE, retinal microaneurysms, epiretinal membrane conjunctivitis, physiopedia, nystagmus-oscillopsia, diplopia, or gaze-evoked tinnitus (GET).

34-35. (canceled)

36. A nucleic acid molecule comprising a sequence that is at least 90%, 95%, 96%, 97%, 98%, or 99% identical, or is identical, to SEQ ID NO: 2.

37-42. (canceled)

43. The nucleic acid molecule of claim 36, wherein:
the nucleic acid comprising SEQ ID NO: 2 is operably linked to regulatory sequences which direct expression of the protein encoded by SEQ ID NO: 2, wherein the regulatory sequences comprise:
a constitutive or tissue specific promoter;
a stuffer sequence upstream of the nucleic acid molecule comprising the nucleic acid sequence of SEQ ID NO: 2 and downstream of an AAV ITR positioned 5′ to the nucleic acid sequence of SEQ ID NO: 2; and
an intron sequence that is downstream of SEQ ID NO: 2 and upstream of an AAV ITR positioned 3′ to the nucleic acid sequence of SEQ ID NO: 2.

44-54. (canceled)

55. A method of producing an AAV particle of claim 1, the method comprising contacting a cell with a nucleic acid molecule of claim 36 to produce the AAV.

56-65. (canceled)

66. The AAV of claim 1, wherein the active fragment consists essentially of residues 1-359 of SEQ ID NO: 1, residues 1-313 of SEQ ID NO: 1, residues 1-219 of SEQ ID NO: 1, residues 1-73 of SEQ ID NO: 1, residues 312-595 of SEQ ID NO: 1, residues 479-595 of SEQ ID NO: 1, or residues 503-595 of SEQ ID NO: 1, or combination thereof.
